SEQUENCE LISTING (25 sequences)

SEQ ID NO: 1 - DNA, 19 bp, synthetic construct
  misc_feature 1..19: note = Synthetic: Pgp3-pgp4-F
  source 1..19: mol_type = other DNA
agttcgaattacggggttt

SEQ ID NO: 2 - DNA, 32 bp, synthetic construct
  misc_feature 1..32: note = Synthetic: His-RBS-R
  source 1..32: mol_type = other DNA
ggtaggtcctaactattcatttcacttttctc

SEQ ID NO: 3 - DNA, 29 bp, synthetic construct
  misc_feature 1..29: note = Synthetic: RBS-His-F
  source 1..29: mol_type = other DNA
atagttaggacctaccggtatgggcagca

SEQ ID NO: 4 - DNA, 35 bp, synthetic construct
  misc_feature 1..35: note = Synthetic: GrgA-67-R
  source 1..35: mol_type = other DNA
attacggatacttaacttatatccttctctagatg

SEQ ID NO: 5 - DNA, 30 bp, synthetic construct
  misc_feature 1..30: note = Synthetic: GrgA-67-F
  source 1..30: mol_type = other DNA
gttaagtatccgtaattcgaaacacttgtc

SEQ ID NO: 6 - DNA, 19 bp, synthetic construct
  misc_feature 1..19: note = Synthetic: Pgp4-pgp3-R
  source 1..19: mol_type = other DNA
accccgtaattcgaacttt

SEQ ID NO: 7 - DNA, 53 bp, synthetic construct
  misc_feature 1..53: note = Synthetic: GrgA67_IBS1/2
  source 1..53: mol_type = other DNA
aaaaaagcttataattatccttaataagctatccagtgcgcccagatagggtg

SEQ ID NO: 8 - DNA, 28 bp, synthetic construct
  misc_feature 1..28: note = Synthetic: Universal primer
  source 1..28: mol_type = other DNA
cgaaattagaaacttgcgttcagtaaac

SEQ ID NO: 9 - DNA, 49 bp, synthetic construct
  misc_feature 1..49: note = Synthetic: GrgA67_EBS2
  source 1..49: mol_type = other DNA
tgaacgcaagtttctaatttcggttcttatccgatagaggaaagtgtct

SEQ ID NO: 10 - DNA, 60 bp, synthetic construct
  misc_feature 1..60: note = Synthetic: GrgA67_EBS1/delta
  source 1..60: mol_type = other DNA
cagattgtacaaatgtggtgataacagataagtctatccatttaacttacctttctttgt

SEQ ID NO: 11 - DNA, 24 bp, synthetic construct
  misc_feature 1..24: note = Synthetic: gA
  source 1..24: mol_type = other DNA
actgttattacatctagagaagga

SEQ ID NO: 12 - DNA, 20 bp, synthetic construct
  misc_feature 1..20: note = Synthetic: gB
  source 1..20: mol_type = other DNA
agacattggagctacaggtg

SEQ ID NO: 13 - DNA, 20 bp, synthetic construct
  misc_feature 1..20: note = Synthetic: pT
  source 1..20: mol_type = other DNA
taatacgactcactataggg

SEQ ID NO: 14 - DNA, 20 bp, synthetic construct
  misc_feature 1..20: note = Synthetic: gU
  source 1..20: mol_type = other DNA
gaaataggctatgcaactcg

SEQ ID NO: 15 - DNA, 7858 bp, synthetic construct
  misc_feature 1..7858: note = Synthetic: pTargetron-aadA-gagA67
  source 1..7858: mol_type = other DNA
gaattccggatgagcattcatcaggcgggcaagaatgtgaataaaggccggataaaacttgtgcttatttttctttacggtctttaaaaaggccgtaatatccagctgaacggtctggttataggtacattgagcaactgactgaaatgcctcaaaatgttctttacgatgccattgggatatatcaacggtggtatatccagtgatttttttctccattttagcttccttagctcctgaaaatctcgataactcaaaaaatacgcccggtagtgatcttatttcattatggtgaaagttggaacctcttacgtgccgatcaacgtctcattttcgccaaaagttggcccagggcttcccggtatcaacagggacaccaggatttatttattctgcgaagtgatcttccgtcacaggtatttattcggcgcaaagtgcgtcgggtgatgctgccaacttactgatttagtgtatgatggtgtttttgaggtgctccagtggcttctgtttctatcagctgtccctcctgttcagctactgacggggtggtgcgtaacggcaaaagcaccgccggacatcagcgctagcggagtgtatactggcttactatgttggcactgatgagggtgtcagtgaagtgcttcatgtggcaggagaaaaaaggctgcaccggtgcgtcagcagaatatgtgatacaggatatattccgcttcctcgctcactgactcgctacgctcggtcgttcgactgcggcgagcggaaatggcttacgaacggggcggagatttcctggaagatgccaggaagatacttaacagggaagtgagagggccgcggcaaagccgtttttccataggctccgcccccctgacaagcatcacgaaatctgacgctcaaatcagtggtggcgaaacccgacaggactataaagataccaggcgtttccccctggcggctccctcgtgcgctctcctgttcctgcctttcggtttaccggtgtcattccgctgttatggccgcgtttgtctcattccacgcctgacactcagttccgggtaggcagttcgctccaagctggactgtatgcacgaaccccccgttcagtccgaccgctgcgccttatccggtaactatcgtcttgagtccaacccggaaagacatgcaaaagcaccactggcagcagccactggtaattgatttagaggagttagtcttgaagtcatgcgccggttaaggctaaactgaaaggacaagttttggtgactgcgctcctccaagccagttacctcggttcaaagagttggtagctcagagaaccttcgaaaaaccgccctgcaaggcggttttttcgttttcagagcaagagattacgcgcagaccaaaacgatctcaagaagatcatcttattaatcagataaaatatttctagctagatttcagtgcaatttatctcttcaaatgtagcacctgaagtcagccccatacgatataagttgtaattctcatgtttgacagcttatcatcgataagctcaaggagatggcgcccaacagtcccccggccacggggcctgccaccatacccacgccgaaacaagcgctcatgagcccgaagtggcgagcccgatcttccccatcggtgatgtcggcgatataggcgccagcaaccgcacctgtggcgccggtgatgccggccacgatgcgtccggcgtagaggatcgagatctcgatcccgcgaaattaatacgactcactataggggaattgtgagcggataacaattcccctctagaggtaccggatcctccaaattattccttacatgaattttttgtctgagcgactttctcccattgaaaaagattttcttaaacaaaacgtgctttacttcttgcagaaaaatcggtaaacttgccgtttcgtctaggcagactcgtccgcgtcttttttcaaaactccctttttaggaagtttttgaaggcgttcctcagattttcccgagttggaggagactggccggcactacaagctcatatcaaggtaaggaaagatttccaagcttataattatccttaataagctatccagtgcgcccagatagggtgttaagtcaagtagtttaaggtactactctgtaagataacacagaaaacagccaacctaaccgaaaagcgaaagctgatacgggaacagagcacggttggaaagcgatgagttacctaaagacaatcgggtacgactgagtcgcaatgttaatcagatataaggtataagttgtgtttactgaacgcaagtttctaatttcggttcttatccgatagaggaaagtgtctgaaacctctagtacaaagaaaggtaagttaaatggatagacttatctgttatcaccacatttgtacaatctgtaggagaacctatgggaacgaaacgaaagcgatgccgagaatctgaatttaccaagacttaacactaactggggataccctaaacaagaatgcctaatagaaaggaggaaaaaggctatagcactagagcttgaaaatcttgcaagggtacggagtactcgtagtagtctgagaagggtaacgccctttacatggcaaaggggtacagttattgtgtactaaaattaaaaattgattagggaggaaaacctcaaaatgaaaccaacaatggcaattttagaaagaatcagtaaaaattcacaagaaaatatagacgaagtttttacaagactttatcgttatcttttacgtccagatatttattacgtggcgacgcgttgcctgacgatgcgtggagaccgaaaccttgcgctcgttcgccagccaggacagaaatgcctcgacttcgctgctgcccaaggttgccgggtgacgcacaccgtggaaacggatgaaggcacgaacccagtggacataagcctgttcggttcgtaagctgtaatgcaagtagcgtatgcgctcacgcaactggtccagaaccttgaccgaacgcagcggtggtaacggcgcagtggcggttttcatggcttgttatgactgtttttttggggtacagtctatgcctcgggcatccaagcagcaagcgcgttacgccgtgggtcgatgtttgatgttatggagcagcaacgatgttacgcagcagggcagtcgccctaaaacaaagttaaacatcatgagggaagcggtgatcgccgaagtatcgactcaactatcagaggtagttggcgtcatcgagcgccatctcgaaccgacgttgctggccgtacatttgtacggctccgcagtggatggcggcctgaagccacacagtgatattgatttgctggttacggtgaccgtaaggcttgatgaaacaacgcggcgagctttgatcaacgaccttttggaaacttcggcttcccctggagagagcgagattctccgcgctgtagaagtcaccattgttgtgcacgacgacatcattccgtggcgttatccagctaagcgcgaactgcaatttggagaatggcagcgcaatgacattcttgcaggtatcttcgagccagccacgatcgacattgatctggctatcttgctgacaaaagcaagagaacatagcgttgccttggtaggtccagcggcggaggaactctttgatccggttcctgaacaggatctatttgaggcgctaaatgaaaccttaacgctatggaactcgccgcccgactgggctggcgatgagcgaaatgtagtgcttacgttgtcccgcatttggtacagcgcagtaaccggcaaaatcgcgccgaaggatgtcgctgccgactgggcaatggagcgcctgccggcccagtatcagcccgtcatacttgaagctagacaggcttatcttggacaagaagaagatcgcttggcctcgcgcgcagatcagttggaagaatttgtccactacgtgaaaggcgagatcaccaaggtagtcggcaaataatgtctaacaattcgttcaagccgacgccgcttcgcggcgcggcttaactcaagcgttagatgcactaagcacataattgctcacagccaaactatcaggcccgggacgcgttgggaaatggcaatgatagcgaaacaacgtaaaactcttgttgtatgctttcattgtcatcgtcacgtgattcataaacacaagtgaatgtcgacagtgaatttttacgaacgaacaataacagagccgtatactccgagaggggtacgtacggttcccgaagagggtggtgcaaaccagtcacagtaatgtgaacaaggcggtacctccctacttcaccatatcattttctgcagccccctagaaataattttgtttaactttaagaaggagatatacatatatggctagatcgtccattccgacagcatcgccagtcactatggcgtgctgctagcgctatatgcgttgatgcaatttctatgcactcgtagtagtctgagaagggtaacgccctttacatggcaaaggggtacagttattgtgtactaaaattaaaaattgattagggaggaaaacctcaaaatgaaaccaacaatggcaattttagaaagaatcagtaaaaattcacaagaaaatatagacgaagtttttacaagactttatcgttatcttttacgtccagatatttattacgtggcgtatcaaaatttatattccaataaaggagcttccacaaaaggaatattagatgatacagcggatggctttagtgaagaaaaaataaaaaagattattcaatctttaaaagacggaacttactatcctcaacctgtacgaagaatgtatattgcaaaaaagaattctaaaaagatgagacctttaggaattccaactttcacagataaattgatccaagaagctgtgagaataattcttgaatctatctatgaaccggtattcgaagatgtgtctcacggttttagacctcaacgaagctgtcacacagctttgaaaacaatcaaaagagagtttggcggcgcaagatggtttgtggagggagatataaaaggctgcttcgataatatagaccacgttacactcattggactcatcaatcttaaaatcaaagatatgaaaatgagccaattgatttataaatttctaaaagcaggttatctggaaaactggcagtatcacaaaacttacagcggaacacctcaaggtggaattctatctcctcttttggccaacatctatcttcatgaattggataagtttgttttacaactcaaaatgaagtttgaccgagaaagtccagaaagaataacacctgaatatcgggagctccacaatgagataaaaagaatttctcaccgtctcaagaagttggagggtgaagaaaaagctaaagttcttttagaatatcaagaaaaacgtaaaagattacccacactcccctgtacctcacagacaaataaagtattgaaatacgtccggtatgcggacgacttcattatctctgttaaaggaagcaaagaggactgtcaatggataaaagaacaattaaaactttttattcataacaagctaaaaatggaattgagtgaagaaaaaacactcatcacacatagcagtcaacccgctcgttttctgggatatgatatacgagtaaggagatctggaacgataaaacgatctggtaaagtcaaaaagagaacactcaatgggagtgtagaactccttattcctcttcaagacaaaattcgtcaatttatttttgacaagaaaatagctatccaaaagaaagatagctcatggtttccagttcacaggaaatatcttattcgttcaacagacttagaaatcatcacaatttataattctgaactccgcgggatttgtaattactacggtctagcaagtaattttaaccagctcaattattttgcttatcttatggaatacagctgtctaaaaacgatagcctccaaacataagggaacactttcaaaaaccatttccatgtttaaagatggaagtggttcgtgggggatcccgtatgagataaagcaaggtaagcagcgccgttattttgcaaattttagtgaatgtaaatccccttatcaatttacggatgagataagtcaagctcctgtattgtatggctatgcccggaatactcttgaaaacaggttaaaagctaaatgttgtgaattatgtgggacgtctgatgaaaatacttcctatgaaattcaccatgtcaataaggtcaaaaatcttaaaggcaaagaaaaatgggaaatggcaatgatagcgaaacaacgtaaaactcttgttgtatgctttcattgtcatcgtcacgtgattcataaacacaagtgaatgtcgagcacccgttctcggagcactgtccgaccgctttggccgccgcccagtcctgctcgcttcgctacttggagccactatcgactacgcgatcatggcgaccacacccgtcctgtggatcgccaagctcgccgatggtagtgtggggtctccccatgcgagagtagggaactgccaggcatcaaataaaacgaaaggctcagtcgaaagactgggcctttcgttttatctgttgtttgtcggtgaacgctctcctgagtaggacaaatccgccgggagcggatttgaacgttgcgaagcaacggcccggagggtggcgggcaggacgcccgccataaactgccaggcatcaaattaagcagaaggccatcctgacggatggcctttttgcgtttctacaaactcttcctgtcgtcatatctacaagccatccccccacagatacggtaaactagcctcgtttttgcatcaggaaagcagaacgccatgagcggcctcatttcttattctgagttacaacagtccgcaccgctgtccggtagctccttccggtgggcgcggggcatgactatcgtcgccgcacttatgactgtcttctttatcatgcaactcgtaggacaggtgccggcagcgcccaacagtcccccggccacggggcctgccaccatacccacgccgaaacaagcgccctgcaccattatgttccggatctgcatcgcaggatgctgctggctaccctgtggaacacctacatctgtattaacgaagcgctaaccgtttttatcaggctctgggaggcagaataaatgatcatatcgtcaattattacctccacggggagagcctgagcaaactggcctcaggcatttgagaagcacacggtcacactgcttccggtagtcaataaaccggtaaaccagcaatagacataagcggctatttaacgaccctgccctgaaccgacgaccgggtcgaatttgctttcgaatttctgccattcatccgcttattatcacttattcaggcgtagcaccaggcgtttaagggcaccaataactgccttaaaaaaattacgccccgccctgccactcatcgcagtactgttgtaattcattaagcattctgccgacatggaagccatcacagacggcatgatgaacctgaatcgccagcggcatcagcaccttgtcgccttgcgtataatatttgcccatggtgaaaacgggggcgaagaagttgtccatattggccacgtttaaatcaaaactggtgaaactcacccagggattggctgagacgaaaaacatattctcaataaaccctttagggaaataggccaggttttcaccgtaacacgccacatcttgcgaatatatgtgtagaaactgccggaaatcgtcgtggtattcactccagagcgatgaaaacgtttcagtttgctcatggaaaacggtgtaacaagggtgaacactatcccatatcaccagctcaccgtctttcattgccatacg

SEQ ID NO: 16 - DNA, 12270 bp, synthetic construct
  misc_feature 1..12270: note = Synthetic: pTRL2-peig
  source 1..12270: mol_type = other DNA
atggaaaatagaggtaccatggtctctgaacttatcaaagaaaatatgcacatgaaattatacatggaaggcactgtcaacaatcatcactttaaatgcacctctgaaggtgaaggcaaaccgtatgaaggaactcaaacaatgcgcattaaagctgtagaaggaggtcctcttccgtttgctttcgatatcctggcaacttctttcatgtacggttctaaaaccttcatcaatcatacgcaaggcatccctgatttctttaaacagtcttttccggaaggcttcacttgggaacgcgtaactacatatgaagatggcggggtcctgaccgcgacgcaagatacatctctgcaggatggatgtcttatctacaacgttaaaatccgtggggtgaattttccatctaacggacctgttatgcaaaagaaaactctggggtgggaagcgtctactgaaacattatatccagccgatggaggtcttgaaggtcgtgcggatatggccctgaaattagtgggcgggggacaccttatttgtaatctgaaaaccacgtatcgctctaaaaaaccggctaaaaacctgaaaatgccaggtgtatattacgtcgatcgtcgcttagaacgtatcaaagaagcagataaagaaacttacgttgaacagcatgaagttgctgtggcacgttactgcgatttaccttctaaacttggacaccgctaaggcgccagggttccgatttagtgctttacggcacctcgaccccaaaaaacttgattagggtgatggttcacgtagtgggccatcgccctgatagacggtttttcgccctttgacgttggagtccacgttctttaatagtggactcttgttccaaactggaacaacactcaaccctatctcggtctattcttttgatttataagggattttgccgatttcggcctattggttaaaaaatgagctgatttaacaaaaatttaacgcgaattttaacaaaatattaacgcttacaatttcaggtggcacttttcggggaaatgtgcgcggaacccctatttgtttatttttctaaatacattcaaatatgtatccgctcatgagacaataaccctgataaatgcttcaataatattgaaaaaggaagagtatgagtattcaacatttccgtgtcgcccttattcccttttttgcggcattttgccttcctgtttttgctcacccagaaacgctggtgaaagtaaaagatgctgaagatcagttgggtgcacgagtgggttacatcgaactggatctcaacagcggtaagatccttgagagttttcgccccgaagaacgttttccaatgatgagcacttttaaagttctgctatgtggcgcggtattatcccgtattgacgccgggcaagagcaactcggtcgccgcatacactattctcagaatgacttggttgagtactcaccagtcacagaaaagcatcttacggatggcatgacagtaagagaattatgcagtgctgccataaccatgagtgataacactgcggccaacttacttctgacaacgatcggaggaccgaaggagctaaccgcttttttgcacaacatgggggatcatgtaactcgccttgatcgttgggaaccggagctgaatgaagccataccaaacgacgagcgtgacaccacgatgcctgtagcaatggcaacaacgttgcgcaaactattaactggcgaactacttactctagcttcccggcaacaattgatagactggatggaggcggataaagttgcaggaccacttctgcgctcggcccttccggctggctggtttattgctgataaatctggagccggtgagcgtggctctcgcggtatcattgcagcactggggccagatggtaagccctcccgtatcgtagttatctacacgacggggagtcaggcaactatggatgaacgaaatagacagatcgctgagataggtgcctcactgattaagcattggtaggaattaatgatgtctcgtttagataaaagtaaagtgattaacagcgcattagagctgcttaatgaggtcggaatcgaaggtttaacaacccgtaaactcgcccagaagctaggtgtagagcagcctacattgtattggcatgtaaaaaataagcgggctttgctcgacgccttagccattgagatgttagataggcaccatactcacttttgccctttagaaggggaaagctggcaagattttttacgtaataacgctaaaagttttagatgtgctttactaagtcatcgcgatggagcaaaagtacatttaggtacacggcctacagaaaaacagtatgaaactctcgaaaatcaattagcctttttatgccaacaaggtttttcactagagaatgcattatatgcactcagcgcagtggggcattttactttaggttgcgtattggaagatcaagagcatcaagtcgctaaagaagaaagggaaacacctactactgatagtatgccgccattattacgacaagctatcgaattatttgatcaccaaggtgcagagccagccttcttattcggccttgaattgatcatatgcgattagaaaaacaacttaaatgtgaaagtgggtcttaaaagcagcataacctttttccgtgatggtaacttcactagtttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgtccttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgacccgacaccatcgaatggccagatgattaattcctaatttttgttgacactctatcattgatagagttattttaccactccctatcagtgatagagaaaagtgaaatgaatagttaggacctaccggtatgggcagcagccatcatcatcatcatcacagcagcggcctggtgccgcgcggcagccatatgtattttacaagagatccagtcatagagactgttattacatctagagaaggatataagttaagtatccgtaattcgaaacacttgtcccaagatccttttgtcgttgaggctatagaggttgtccgtttaggagggactagttttttccgtaattgtgatcatagtaagccgtttttactgccagcatctgattatgaagtgatggaaatccgggatgctaaaatcaaccttaaagctgttggtttagatcgtggagtcaagattgttggtagtcgggaagctttactaaagatgccgaaggtggctccaatagtttctgtatcggaagataatacgattgtttctgaagaagaggtagttgcagactctactgttgcagctcccgcttctacacctgtagctccaatgtctaagaaagagagacgaaaagagtttaagaatgagaaatggaaggataagaaaaaacaaggacgtcgtcgaaatagtaaagagattgccgatgctgttggatcttctcaagagatgatcgacaccgtagcagaggaatgtttgcaagagtcctcttctgaggaaggcgatttcagtgagcgacggttttctttgattcctcctcctactcgattgatttctgatggtccagaagaacccgaggaagagtctcagcctgtgacttcagtggatttaaatgagtctctaaacgctttagtcagcgaaagttgcaatgttattgagtctattttagccgatgaggacacggttgtttttactaaagaaaaagatcaaactgctgaagaatctcaagagcagccaagtctttcattagaagaaactcctgttcatgacagtatttcttcagaagagtaaagtgaaaaatggcgcacattgtgcgacattttttttgtctgccgtttaccgctactgcgtcacggatctccacgcgccctgtagcggcgcattaagcgcggcgggtgtggtggttacgcgcagcgtgaccgctacacttgccagcgccctagcgcccgctcctttcgctttcttcccttcctttctcgccacgttcgccggctttccccgtcaagctctaaagggcccaagaggtaagtcctctagtacaaacacccccaatattgtgatataattaaaattatattcatattctgttgccagaaaaaacacttttaggctatattagagccatcttctttgaagcgttgtcttctcgagaagatttatcgtacgcaaatatcatctttgcggttgcgtgtcctgtgaccttcattatgtcggagtctgagcaccctaggcgtttgtactccgtcacagcggttgctcgaagcacgtgcggggttatcttaaaagggattgcagcttgtagtcctgcttgagagaacgtgcgggcgatttgccttaaccccaccatttttccggagcgagttacgaagacaaaacctcttcgttgaccgatgtactcttgtagaaagtgcataaacttctgaggataagttataataatcctcttttctgtctgacggttcttaagctgggagaaagaaatggtagcttgttggaaacaaatctgactaatctccaagcttaagacttcagaggagcgtttacctccttggagcattgtctgggcgatcaaccaatcccgggcattgattttttttagctcttttaggaaggacgctgtttgcaaactgttcatcgcatctgtttttactatttccctggttttaaaaaatgttcgactattttcttgtttagaaggttgcgctatagcgactattccttgagtcatcctgtttaggaatcttgttaaggaaatatagcttgctgctcgaacttgtttagtaccttcggtccaagaagtcttggcagaggaaacttttttaatcgcatctagaattagattatgatttaaaagggaaaactcttgcagattcatatccaaggacaatagaccaatcttttctaaagacaaaaaagatcctcgatatgatctacaagtatgtttgttgagtgatgcggtccaatgcataataacttcgaataaggagaagcttttcatgcgtttccaataggattcttggcgaatttttaaaacttcctgataagacttttcgctatattctaacgacatttcttgctgcaaagataaaatccctttacccatgaaatccctcgtgatataacctatccgtaaaatgtcctgattagtgaaataatcaggttgttaacaggatagcacgctcggtatttttttatataaacatgaaaactcgttccgaaatagaaaatcgcatgcaagatatcgagtatgcgttgttaggtaaagctctgatatttgaagactctactgagtatattctgaggcagcttgctaattatgagtttaagtgttctcatcataaaaacatattcatagtatttaaatacttaaaagacaatggattacctataactgtagactcggcttgggaagagcttttgcggcgtcgtatcaaagatatggacaaatcgtatctcgggttaatgttgcatgatgctttatcaaatgacaagcttagatccgtttctcatacggttttcctcgatgatttgagcgtgtgtagcgctgaagaaaatttgagtaatttcattttccgctcgtttaatgagtacaatgaaaatccattgcgtagatctccgtttctattgcttgagcgtataaagggaaggcttgacagtgctatagcaaagactttttctattcgcagcgctagaggccggtctatttatgatatattctcacagtcagaaattggagtgctggctcgtataaaaaaaagacgagcaacgttctctgagaatcaaaattctttctttgatgccttcccaacaggatacaaggatattgatgataaaggagttatcttagctaaaggtaatttcgtgattatagcagctaggccatctatagggaaaactgctttagctatagacatggcgataaatcttgcggttactcaacagcgtagagttggtttcctatctctagaaatgagcgcaggtcaaattgttgagcggattattgctaatttaacaggaatatctggtgaaaaattacaaagaggggatctctctaaagaagaattattccgagtagaagaagctggagaaacagttagagaatcacatttttatatctgcagtgatagtcagtataagcttaatttaatcgcgaatcagatccggttgctgagaaaggaagatcgagtagacgtaatatttatcgattacttgcagttgatcaactcatcggttggagaaaatcgtcaaaatgaaatagcagatatatctagaaccttaagaggtttagcctcagagctaaacattcctatagtttgtttatcccaactatctagaaaagttgaggatagagcaaataaagttcccatgctttcagatttgcgagacagcggtcaaatagagcaagacgcagatgtgattttgtttatcaataggaaggaatcgtcttctaattgtgagataactgttgggaaaaatagacatggatcggttttctcttcggtattacatttcgatccaaaaattagtaaattctccgctattaaaaaagtatggtaaattatagtaactgccacttcatcaaaagtcctatccaccttgaaaatcagaagtttggaagaagacctggtcaatctattaagatatctcccaaattggctcaaaatgggatggtagaagttataggtcttgattttctttcatctcattaccatgcattagcagctatccaaagattgctgactgcaacgaattacaaggggaacacaaaaggggttgttttatccagagaatcaaatagttttcaatttgaaggatggataccaagaatccgttttacaaaaactgaattcttagaggcttatggagttaagcggtataaaacatccagaaataagtatgagtttagtggaaaagaagctgaaactgctttagaagccttataccatttaggacatcaaccgtttttaatagtggcaactagaactcgatggactaatggaacacaaatagtagaccgttaccaaactctttctccgatcattaggatttacgaaggatgggaaggtttaactgacgaagaaaatatagatatagacttaacaccttttaattcaccatctacacggaaacataaagggttcgttgtagagccatgtcctatcttggtagatcaaatagaatcctactttgtaatcaagcctgcaaatgtataccaagaaataaaaatgcgcttcccaaatgcatcaaagtatgcttacacatttatcgactgggtgattacagcagctgcgaaaaagagacgaaaattaactaaggataattcttggccagaaaacttgttcttaaacgttaacgttaaaagtcttgcatatattttaaggatgaatcggtacatttgtacaaggaactggaaaaaaatcgagttagctatcgataaatgtatagaaatcgccattcagcttggttggttatctagaagaaaacgcattgaatttctggattcttctaaactctctaaaaaagaaattctatatctaaataaagagcgttttgaagaaataactaagaaatctaaagaacaaatggaacaattagaacaagaatctattaattaatagcaaacttgaaactaaaaacctaatttatttaaagctcaaaataaaaaagagttttaaaatgggaaattctggtttttatttgtataacactcaaaactgcgtctttgctgataatatcaaagttgggcaaatgacagagccgctcaaggaccagcaaataatccttgggacaacatcaacacctgtcgcagccaaaatgacagcttctgatggaatatctttaacagtctccaataatccatcaaccaatgcttctattacaattggtttggatgcggaaaaagcttaccagcttattctagaaaagttgggagatcaaattcttggtggaattgctgatactattgttgatagtacagtccaagatattttagacaaaatcacaacagacccttctctaggtttgttgaaagcttttaacaactttccaatcactaataaaattcaatgcaacgggttattcactcccaggaacattgaaactttattaggaggaactgaaataggaaaattcacagtcacacccaaaagctctgggagcatgttcttagtctcagcagatattattgcatcaagaatggaaggcggcgttgttctagctttggtacgagaaggtgattctaagccctacgcgattagttatggatactcatcaggcgttcctaatttatgtagtctaagaaccagaattattaatacaggattgactccgacaacgtattcattacgtgtaggcggtttagaaagcggtgtggtatgggttaatgccctttctaatggcaatgatattttaggaataacaaatacttctaatgtatcttttttggaggtaatacctcaaacaaacgcttaaacaatttttattggatttttcttataggttttatatttagagaaaaaagttcgaattacggggtttgttatgcaaaataaaagcaaagtgagggacgattttattaaaattgttaaagatgtgaaaaaagatttccccgaattagacctaaaaatacgagtaaacaaggaaaaagtaactttcttaaattctcccttagaactctaccataaaagtgtctcactaattctaggactgcttcaacaaatagaaaactctttaggattattcccagactctcctgttcttgaaaaattagaggataacagtttaaagctaaaaaaggctttgattatgcttatcttgtctagaaaagacatgttttccaaggctgaatagataacttactctaacgttggagttgatttgcacaccttagttttttgctcttttaagggaggaactggaaaaacaacactttctctaaacgtgggatgcaacttggcccaatttttagggaaaaaagtgttacttgctgacctagacccgcaatccaatttatcttctggattgggggctagtgtcagaagtaaccaaaaaggcttacacgacatagtatacacatcaaacgatttaaaatcaatcatttgcgaaacaaaaaaagatagtgtggacctaattcctgcatcatttttatccgaacagtttagagaattggatattcatagaggacctagtaacaacttaaagttatttctgaatgagtactgcgctcctttttatgacatctgcataatagacactccacctagcctaggagggttaacgaaagaagcttttgttgcaggagacaaattaattgcttgtttaactccagaacctttttctattctagggttacaaaagatacgtgaattcttaagttcggtcggaaaacctgaagaagaacacattcttggaatagctttgtctttttgggatgatcgtaactcgactaaccaaatgtatatagacattatcgagtctatttacaaaaacaagcttttttcaacaaaaattcgtcgagatatttctctcagccgttctcttcttaaagaagattctgtagctaatgtctatccaaattctagggccgcagaagatattctgaagttaacgcatgaaatagcaaatattttgcatatcgaatatgaacgagattactctcagaggacaacgtgaacaaactaaaaaaagaagcgaatgtcttttttaaaaaaaatcaaactgccgcttctttagattttaagaagacgcttccttccattgaactattctcagcaactttgaattctgaggaaagtcagagtttggatcaattatttttatcagagtcccaaaactattcggatgaagaattttatcaagaagacatcctagcggtaaaactgcttactggtcagataaaatccatacagaagcaacacgtacttcttttaggagaaaaaatctataatgctagaaaaatcctgagtaaggatcacttctcctcaacaactttttcatcttggatagagttagtttttagaactaagtcttctgcttacaatgctcttgcatattacgagctttttataaacctccccaaccaaactctacaaaaagagtttcaatcgatcccctataaatccgcatatattttggccgctagaaaaggcgatttaaaaaccaaggtcgatgtgatagggaaagtatgtggaatgtcgaactcatcggcgataagggtgttggatcaatttcttccttcatctagaaacaaagacgttagagaaacgatagataagtctgattcagagaagaatcgccaattatctgatttcttaatagagatacttcgcatcatgtgttccggagtctctttgccctcctataacgaaaatcttctacaacagctttttgaactttttaagcaaaagagctgatcctccgtcagctcatatatatatctattatatatatatatttagggatttgattttacgagagagatttgcaactcttggtggtagactttgcaactcttggtggtagactttgcaactcttggtggtagactttgcaactcttggtggtagacttggtcataatggacttttgttgaaaaatttcttaaaatcttagagctccgattttgaatagctttggttaagaaaatgggctcgatggctttccataaaagtaggttgttcttaacttttggggacgcgtcggaaatttggttatctactttatctcatctaactagaaaaaattatgcgtctgggattaactttcttgtttctttagagattctggatttatcggaaaccttgataaaggctatttctcttgaccacagcgaatctttgtttaaaatcaagtctctagatgtttttaatggaaaagtcgtttcagaggcctctaaacaggctagagcggcatgctacatatctttcacaaagtttttgtatagattgaccaagggatatattaaacccgctattccattgaaagattttggaaacactacattttttaaaatccgagacaaaatcaaaacagaatcgatttctaagcaggaatggacagttttttttgaagcgctccggatagtgaattatagagactatttaatcggtaaattgattgtacaagggatccgtaagttagacgaaattttgtctttgcgcacagacgatctattttttgcatccaatcagatttcctttcgcattaaaaaaagacagaataaagaaaccaaaattctaatcacatttcctatcagcttaatggaggagttgcaaaaatacacttgtgggagaaatgggagagtatttgtttctaaaatagggattcctgtaacaacaagtcaggttgcgcataattttaggcttgcagagttctatagtgctatgaaaaaaaaattactcctagagtacttcgtgcaagcgctttgattcatttaaagcaaataggattaaaagatgaggaaatcatgcgtatttcctgtctttcatcgagacaaagtgtgtgttcttattgttctgggtgtcgacattcttgaacggtggagacggtttcttataatgacaccgactt

SEQ ID NO: 17 - DNA, 27 bp, synthetic construct
  misc_feature 1..27: note = Synthetic: wildtype nucleotide sequence
  source 1..27: mol_type = other DNA
gcattagagcaccatactaaagaagaa

SEQ ID NO: 18 - protein, 9 aa, synthetic construct
  REGION 1..9: note = Synthetic: wildtype amino acid sequence
ALEHHTKEE

SEQ ID NO: 19 - DNA, 27 bp, synthetic construct
  misc_feature 1..27: note = Synthetic: mutant nucleotide sequence
  source 1..27: mol_type = other DNA
gcattatagcccatactcaaagaataa

SEQ ID NO: 20 - protein, 7 aa, synthetic construct
  REGION 1..7: note = Synthetic: mutant amino acid sequence
ALPILKE

SEQ ID NO: 21 - DNA, 15 bp, synthetic construct
  misc_feature 1..15: note = Synthetic
  source 1..15: mol_type = other DNA
ttatccattcgtaat

SEQ ID NO: 22 - protein, 5 aa, synthetic construct
  REGION 1..5: note = Synthetic
LSIRN

SEQ ID NO: 23 - DNA, 15 bp, synthetic construct
  misc_feature 1..15: note = Synthetic
  source 1..15: mol_type = other DNA
ttaagtatccgtaat

SEQ ID NO: 24 - DNA, 15 bp, synthetic construct
  misc_feature 1..15: note = Synthetic
  source 1..15: mol_type = other DNA
ttatccagtgcgccc

SEQ ID NO: 25 - DNA, 15 bp, synthetic construct
  misc_feature 1..15: note = Synthetic
  source 1..15: mol_type = other DNA
acttcacttcgtaat

(19) United States
(12) Patent Application Publication
(10) Pub. No.: US 2025/0186573 A1
(43) Pub. Date: Jun. 12, 2025

(54) ATTENUATED MATURATION-DEFECTIVE CHLAMYDIA VACCINES

(71) Applicant: Rutgers, The State University of New Jersey, New Brunswick, NJ (US)

(72) Inventor: Huizhou FAN, New Brunswick, NJ (US)

(21) Appl. No.: 18/845,468

(22) PCT Filed: Mar. 28, 2023
(86) PCT No.: PCT/US23/16633

§ 371 Date: Sep. 10, 2024

Related U.S. Application Data
(60) Provisional application No. 63/362,120, filed on Mar. 29, 2022.

(51) Int. Cl. A61K 39/118; A61K 39/00; C12N 1/20; C12Q 1/02; C12R 1/01

(52) U.S. Cl. CPC A61K 39/118; C12N 1/20; C12Q 1/02; A61K 2039/522; C12R 2001/01

(57) ABSTRACT

Using a novel dependence on plasmid-mediated expression (DOPE) technology, conditional depletion of GrgA, a Chlamydia-specific protein, has been demonstrated to result in greatly reduced reticulate body (RB) proliferation rate and near complete lack of elementary body (EB) formation, thus disrupting the normal chlamydial developmental cycle. This conditional GrgA-deficient Chlamydia allows study of chlamydial growth and developmental regulation and can be used as the basis of an attenuated, maturation-defective but immunogenic bacteria for use as an avirulent vaccine against Chlamydia.

### CROSS-REFERENCE TO RELATED APPLICATIONS

This application is a National Stage entry of PCT/US23/16633, filed 28 Mar. 2023, which claims benefit of U.S. Patent Application No. 63/362,120, filed 29 Mar. 2022. The entire contents of these applications are hereby incorporated by reference as if fully set forth herein.

### GOVERNMENT FUNDING SUPPORT

This invention was made with government support under grant nos. AI140167 and AI154305, awarded by the National Institutes of Health. The government has certain rights in the invention.

### BACKGROUND OF THE INVENTION

### 1. Field of the Invention

This invention relates to the fields of medicine and biology, and in particular to the study of Chlamydia. Specifically, the invention relates to a method for interrupting the developmental cycle of Chlamydia using a conditional knock-out of grgA, the gene that encodes the GrgA protein to produce attenuated, maturation-defective Chlamydia suitable for use as a vaccine against chlamydial disease in humans and animals.

### 2. Background of the Invention

Chlamydia is the most common sexually transmitted bacterial infection in the world in humans. It can have serious consequences, including discharge, burning sensation, swelling of the testicles, and pelvic inflammatory disease, ectopic pregnancy, and infertility in women and men. Symptoms also can occur in the anus, eyes (sometimes resulting in blindness), throat, and lymph nodes. Many people who are infected with Chlamydia have no symptoms or may have symptoms only after a several-week incubation period. Chlamydia also causes community-acquired respiratory infections in humans and a possible risk factor of cardiovascular diseases and age-related neurodegeneration. Because infected people may not be aware that they are suffering from an infection, a vaccine would be particularly useful.

Chlamydia also is a widespread pathogen in animals, including commercially important livestock, protected wildlife, and other animals, including but not limited to cattle, pigs, sheep, goats, guinea pigs, birds (poultry), cats, mice, rabbits, and snakes. However, there is no Chlamydia vaccine for human use, and the efficacy and safety of animal Chlamydia vaccines remain uncertain. In addition, no antibiotics currently exist that selectively inhibit chlamydiae. Therefore, there is a great need in the art for a vaccine according to this invention.

Chlamydiae are obligate intracellular bacterial parasites and have a unique developmental cycle which includes two cellular forms. The first is the Elementary Body (EB), which is infectious but non-dividing, and can temporarily survive in the extracellular environment. The second is the Reticulate Body (RB), which is proliferative but noninfectious, and replicates inside the host cell. The infectious cycle of the obligate intracellular bacterium Chlamydia is initiated when its EB enters a eukaryotic host cell. Within a vacuole (“inclusion”) in the host cytoplasm, the EB differentiates into the proliferative but noninfectious RB. Following rounds of replication, as RBs accumulate inside the inclusion, RBs redifferentiate back into non-dividing EBs before exiting the host cell to infect other host cells or transmit to a new host. See FIG. 1A. In this figure, EBs and RBs are shown as small and large circles, respectively, as indicated.

The chlamydial developmental cycle is transcriptionally regulated. After EBs enter host cells, early genes are activated during the first few hours enabling primary differentiation into RBs. Starting at around 8 hours post-infection, midcycle genes, representing the vast majority of all chlamydial genes, are expressed enabling RB replication. At around 24 hours post-infection, late genes are activated to enable the secondary differentiation of RBs back into EBs.

As a subunit of the RNA polymerase (RNAP) holoenzyme, sigma factor recognizes and binds specific DNA gene promoter elements allowing RNAP to initiate transcription. Chlamydia encodes three sigma factors termed σ⁶⁶, σ²⁸, and σ⁵⁴. σ⁵ RNAP holoenzyme is involved in the expression of early, mid, and late genes, whereas the σ²⁸ RNAP and σ⁵⁴ RNAP are responsible for transcribing only a subset of late or mid-late genes. A small number of genes have tandem promoters, and their expression are regulated by multiple sigma factors.

Understanding the chlamydial developmental mechanism has been hampered by the lack of a robust genetics tool for knocking out essential genes. Therefore, it would be important to develop convenient methods for knocking out these genes in order to identify their biological functions in chlamydial growth and development.

### SUMMARY OF THE INVENTION

Accordingly, the disclosure here shows the production of a Chlamydia knock-out that does not express grgA and is maturation deficient, producing an avirulent bacterium that can be used for research and for vaccine production. Specifically, the invention relates to a Chlamydia knock-out that does not express GrgA. Preferably, the Chlamydia knock-out is selected from the group consisting of C. trachomatis, C. pneumoniae, C. psittaci, C. muridarum, C. suis, C. abortus, C. felis, C. pecorum, C. ibidis, C. avium, C. gallinacea, and the like. Most preferably, the Chlamydia knock-out is selected from the group consisting of C. trachomatis, C. pneumoniae, and C. psittaci to prevent human chlamydial diseases and zoonotic chlamydial diseases.

The invention also relates to a vaccine comprising the Chlamydia knock-out described above and a pharmaceutically acceptable carrier. The invention also relates to a method of stimulating an immune response to Chlamydia in a subject in need, comprising: administering the above vaccine to the subject. The invention also relates to a method of producing neutralizing antibodies to Chlamydia in a subject in need, comprising: administering the vaccine above to the subject.

In another aspect, the invention relates to a method of determining whether a bacterial gene in a bacterial cell is essential to its growth or development, comprising: (a) determining the growth or development of the bacterial cell when the gene is intact and expressed; (b) disrupting the gene and determining the growth or development of the bacterial cell when the gene is not functional; (c) introducing into the bacterial cell with the bacterial gene disrupted a plasmid that contains an inducible version of the bacterial gene and determining the growth or development of the bacterial cell when the bacterial gene is induced and when the bacterial gene is not induced. Preferably, the bacterial cell is a chlamydia spp.

### BRIEF SUMMARY OF THE DRAWINGS

FIG. 1A shows the development cycle of wild type Chlamydia.

FIG. 1B shows the changed cycle of the grgA knock-out Chlamydia mutant.

FIG. 2 shows the domain structure of the GrgA protein.

FIG. 3 shows the rationale of the DOPE technology, which enables physiological and mechanistic interrogation of chromosome-encoded essential genes. Dark and grey lines signify chromosomal and plasmid sequences, respectively. Half arrows indicate the transcription start sites. EG: essential genes; iEG: inducible EG; chr: chromosome; NA: not applicable; g: growth; d: development.

FIG. 4A through FIG. 4E relate to confirmation of the disruption of the chromosome-coded grgA by group II intron and re-expression of GrgA from a transformed plasmid in the DOPE technology. FIG. 4A is a set of schematic drawings of grgA alleles, locations of intron-target site, diagnostic primers, and size of PCR products obtained with different sets of primers. Abbreviations: itsm: intron target site mutated; Chr: chromosome; bp: base pairs. FIG. 4B is a gel image of PCR products amplified with DNA of wildtype C. trachomatis L2 (L2/cg). L2/cg transformed with the his-grgA-itsm expression plasmid (L2/cg-peig), and L2 with aadA-disrupted chromosomal grgA complemented with peig (L2/cgad-peig) using primer sets shown in FIG. 4A. FIG. 4C presents Sanger sequencing tracings of PCR products showing the intron-target site in L2/cg mutations surrounding this site conferring resistance to intron targeting in peig and grgA-intron joint regions in the chromosome of L2/cg-peig. Wildtype bases and corresponding mutated bases are shown with arrowheads and asterisks, respectively. FIG. 4D is a western blot showing detection only chromosome-encoded GrgA in L2/cg-peig cultured in ATC-free medium and both chromosome-coded GrgA and plasmid-coded His-GrgA in L2/cg-peig cultured in the ATC-containing medium for 14 hours. FIG. 4E is a western blot showing time-dependent loss of plasmid-expressed GrgA in L2/cg-peig upon ATC withdrawal.

FIG. 5A through FIG. 5E provide transmission electron microscopy images (TEM) showing lack of EB formation by L2/cgad-peig cultured in ATC-free medium at various late developmental points. FIG. 5A and FIG. 5B are 35 hours postinoculation TEM images of L2/cgad-peig cultured with ATC-free medium and 1 nM ATC medium, respectively. FIG. 5C and FIG. 5D are 45 hours postinoculation TEM images of L2/cgad-peig cultured with ATC-free medium and 1 nM ATC medium, respectively. FIG. 5E is a 60 hours postinoculation TEM image of L2/cgad-peig cultured with ATC-free medium.

FIG. 6A is a schematic showing TetR repression of grgA as the mechanism of GrgA deficiency in L2/cgad-peig cultured in ATC-free medium. FIG. 6B shows the mutations identified in the tetR gene in the plasmids isolated from L2/cg-peig EBs formed with ATC-free medium. The wildtype nucleotide sequence (SEQ ID NO: 17), the wildtype amino acid sequence (SEQ ID NO:18), the mutant nucleotide sequence (SEQ ID NO:19), and the mutant amino acid sequence (SEQ ID NO:20) are shown. FIG. 6C is a schematic showing the loss in grgA repression L2/cg-peig EBs formed with ATC-free medium.

### DETAILED DESCRIPTION

### 1. Definitions

Unless defined otherwise, all technical and scientific terms used herein have the same meaning as commonly understood by one of ordinary skill in the art. Although various methods and materials similar or equivalent to those described herein can be used in the practice or testing of the present invention, suitable methods and materials are described below. However, the skilled artisan understands that the methods and materials used and described are examples and may not be the only ones suitable for use in the invention. Moreover, as measurements are subject to inherent variability, any temperature, weight, volume, time interval, pH, salinity, molarity or molality, range, concentration and any other measurements, quantities or numerical expressions given herein are intended to be approximate and not exact or critical figures unless expressly stated to the contrary.

The term “about,” as used herein, means plus or minus 20 percent of the recited value, so that, for example, “about 0.125” means 0.125±0.025, and “about 1.0” means 1.0±0.2.

As used herein, the term “Chlamydia species” refers to any species in the genera of Chlamydia or Chlamydophila, including, but not limited to: Chlamydia trachomatis, Chlamydia pneumoniae, Chlamydia psittaci, Chlamydia muridarum, Chlamydia suis, Chlamydophila. abortus, Chlamydophila felis, Chlamydophila pecorum, Chlamydia ibidis, Chlamydia avium, Chlamydia gallinacea, and the like.

As used herein, the term “administering” and its cognates refers to introducing an agent to a subject, and can be performed using any of the various methods or delivery systems for administering agents, pharmaceutical compositions or vaccines known to those skilled in the art. Modes of administering include, but are not limited to, nasal and oral administration or intravenous, subcutaneous, intramuscular or intraperitoneal injections, rectal or vaginal administration by way of suppositories or enema, or local administration directly into or onto a target tissue (such as the eye), or administration by any route or method that delivers a therapeutically effective amount of the drug or vaccine composition to the cells or tissue to which it is targeted.

As used herein, the term “vaccine” means any preparation of biological material that contains or produces an antigenic material that upon administration to a subject provides active acquired immunity to at least the pathogenic organism from which the antigenic material was derived. Vaccines can be delivered prophylactically or therapeutically.

As used herein, the terms “treatment,” “treating,” and the like, as used herein refer to obtaining a desired physiologic effect. “Treatment,” includes: (a) preventing or reducing the likelihood of the condition or disease or symptom thereof from occurring in a subject which may be predisposed to the condition or disease but has not yet been diagnosed as having it; (b) inhibiting the condition or disease or symptom thereof, such as, arresting or reducing its development; and (c) relieving, alleviating or ameliorating the condition or disease or symptom thereof, such as, for example, causing regression of the condition or disease or symptom thereof. Treatment therefore refers to administration for the purposes of therapy. A “therapeutically effective amount” is an amount that produces a physiologic response that ameliorates the infection or a symptom thereof or produces a faster resolution of the infection or a symptom thereof. Thus, this amount also includes an amount that produces prevention or reduction of risk of pathological changes in asymptomatically infected individuals.

As used herein, the terms “prophylactic,” “prophylactically,” and the like refer to a preventative treatment, which can mean a complete or partial prevention of the infection or disease condition. Prophylaxis is a preventative measure taken to reduce the likelihood or severity of a disease or condition, such as infection by Chlamydia spp. A “prophylactically effective amount” is an amount that induces an immune response as described below. Such an amount preferably results in the absence of disease upon subsequent exposure or infection, a milder disease upon subsequent exposure or infection, a faster resolution of disease upon subsequent exposure or infection, or a lesser chance of transmission of the organism to a subsequent host.

As used herein, the phrase “induce an immune response,” and its cognates, refers to inducing a physiological response of the subject's immune system to an antigen. An immune response may include an innate immune response, an adaptive immune response, or both. A protective immune response confers immunological cellular memory upon the subject, with the effect that a secondary exposure to the same or a similar antigen is characterized by one or more of the following characteristics: shorter lag phase than the lag phase resulting from exposure to the selected antigen in the absence of prior exposure to the immunizing composition (vaccine); production of antibodies (preferably neutralizing antibodies) which continues for a longer period than production of antibody resulting from exposure to the selected antigen in the absence of prior exposure to the immunizing composition; a change in the type and quality of antibody produced in comparison to the type and quality of antibody produced upon exposure to the selected antigen in the absence of prior exposure to the immunizing composition; an increased average affinity (binding constant) of the antibodies for the antigen in comparison with the average affinity of antibodies for the antigen resulting from exposure to the selected antigen in the absence of prior exposure to the immunizing composition; and/or other characteristics known in the art to characterize a secondary immune response.

As used herein, the term “subject” refers to any animal, including humans. This includes humans, primates, farm animals (including cattle, horses, pigs, sheep, goats, and the like), laboratory animals such as rodents (including mice, rats, guinea pigs, and the like), rabbits and the like, birds (such as chickens, turkeys, geese, ducks and other poultry), companion animals (including dogs, cats, and the like), and reptiles (including snakes, and the like). A “subject in need thereof” refers to any subject suffering from a chlamydial infection, suspected of having a chlamydial infection, or potentially susceptible to developing a chlamydial infection.

### 2. Overview

The developmental cycle of the obligate intracellular bacterium Chlamydia is initiated by its elementary body (EB) entering a eukaryotic host cell. Within a vacuole (inclusion) in the host cytoplasm, the EB differentiates into the proliferative but noninfectious reticulate body (RB). Following rounds of replication, the population of intracellular RBs asynchronously re-differentiates back into the non-dividing EBs before exiting the host cell through either cell lysis or extrusion. Understanding the chlamydial developmental mechanism has been hampered by the lack of a robust genetics tool for knocking out essential genes.

Therefore, a dependence on plasmid-mediated expression (DOPE) technology was developed to allow functional and mechanistic interrogation of chlamydial essential genes. This technology demonstrated that conditional removal of GrgA, a Chlamydia-specific protein, results in a greatly reduced Chlamydia growth rate and near complete lack of RB-to-EB differentiation. Because GrgA is essential for the conversion of RBs to EBs, the GrgA-deficient Chlamydia fully depends on the conditional re-expression of the GrgA protein from an engineered plasmid to complete the cycle to form infectious EBs.

Since the RBs in the mutant Chlamydia are unable to differentiate back into progeny infective EBs, the conditional GrgA-deficient Chlamydia is attenuated and has minimal or no infectivity. The invention therefore relates to a GrgA knock-out, which prevents the infectious form of Chlamydia from forming and methods for its use. This bacterial mutant can be used for studying chlamydial growth and developmental regulation.

In humans and animals, the GrgA-deficient chlamydiae can infect only a limited number of host cells and are incapable of disseminating to additional cells in the same host or transmitting to additional hosts. However, the maturation-defective RBs still elicit host immune response against chlamydiae. GrgA-deficient chlamydiae are ideal attenuated vaccine candidates for human and animals, so the maturation-defective bacteria can be used as an avirulent vaccine.

GrgA is a Chlamydia-specific transcriptional regulator identified through promoter DNA pulldown that binds both σ⁶⁶ and σ²⁸ and activates the transcription of multiple chlamydial genes in vitro and in vivo. RNA-Seq analysis of C. trachomatis conditionally overexpressing GrgA, together with GrgA in vitro transcription assays, has allowed identification of two other transcription factor-encoding genes, euo and hrcA, as members of the GrgA regulon. Both immediate-early genes, euo and hrcA are transcribed during the early phase and midcycle. While Euo is a repressor of chlamydial late genes, HrcA regulates the expression of multiple protein chaperones, which are essential for bacterial growth. These findings suggest that GrgA regulates early chlamydial development.

To further determine the role of GrgA in chlamydial physiology, we attempted but failed to disrupt grgA through group II intron (Targetron™) insertional mutagenesis. Previously, saturated chemical mutagenesis also failed to generate grgA-null mutants. Given that Targetron™ and chemical mutagenesis have been successfully used to disrupt numerous non-essential chlamydial genes, our finding suggest that grgA is an essential gene. In this work, we confirm that grgA is indeed an essential gene by developing and applying a novel genetic tool that we term DOPE (dependence on plasmid-mediated expression). Importantly, we show that GrgA is necessary for RB-to-EB differentiation during the late developmental cycle and is further required for optimal RB growth. This report therefore implicates the requirement of a single chlamydial regulatory factor in the formation of progeny EBs.

### 3. Summary of Results

The chlamydial gene, grgA, was shown to be critical in the developmental cycle of Chlamydia spp.

A Chlamydia trachomatis grgA conditional knock-out was produced.

A new genetics tool was developed to study essential genes in Chlamydia (dependence on plasmid-mediated expression (DOPE) technology).

The DOPE technology showed that the conditional GrgA knock-out leads to both slower Chlamydia growth and lack of RB-to-EB differentiation.

### 4. Embodiments of the Invention

### A. Introduction

Hallmarks of the developmental cycle of the obligate intracellular pathogenic bacterium Chlamydia are the primary differentiation of the infectious elementary body (EB) cell type into the proliferative reticulate body (RB) and the secondary differentiation of RBs back into EBs. The detailed mechanisms regulating these transitions are unclear. In this study, we developed a novel strategy termed DOPE (dependence on plasmid-mediated expression) that allows for the knockdown of essential genes in Chlamydia. Importantly, we demonstrate that GrgA, a Chlamydia-specific transcription factor, is essential for the secondary differentiation of RBs into EBs. Our development of a conditional GrgA-deficient chlamydiae should prove valuable for future studies examining chlamydial growth, development, and pathogenicity. Furthermore, because EB formation is absolutely required for chlamydial dissemination within infected individuals, and for chlamydial transmission to new hosts, our maturation-defective chlamydiae system may serve as an attractive attenuated vaccine methodology for the prevention of chlamydial diseases.

Currently known experimental vaccines have not been effective against Chlamydia spp. in humans and are not approved for use in humans. Pal et al. (2020) have shown that vaccination with the major outer membrane protein (MOMP; a surface-exposed, highly conserved, antigenic protein present in both RB and EB) of Chlamydia muridarum did elicit protection in mice. Human clinical trial data can be viewed online at pubmed.ncbi.nlm.nih.gov/31416692 and in Abraham et al., Lancet 19(10):P1091-1100, 2019. Administration of Chlamydia RB alone is not effective as a vaccine because production of very large amounts of RB is inefficient, and it has not been possible to obtain sufficiently pure RB in the absence of contaminating infectious EB. Therefore, there is a need in the art for new vaccines against this important pathogen.

In bacteria, synthesis of all RNAs is catalyzed by a single RNA polymerase (RNAP). The RNAP holoenzyme (RNAPholo) is comprised of a catalytic core (RNAPcore) and a σ factor, which recognizes the promoter sequence. Transcription factors regulate RNA synthesis by binding DNA, or both DNA and the RNAP. A transcription factor termed GrgA was identified. GrgA is a Chlamydia-specific protein which is expressed in both EBs and RBs. It binds both σ⁶⁶ (the primary σ factor) and σ²⁸ (one of two alternative a factors) in vitro and activates transcription of both σ⁶⁶-dependent genes and σ²⁸-dependent genes in vitro and in vivo. GrgA overexpression leads to increased transcription of numerous genes, including two immediate-early genes coding for transcription factors, termed Euo and HrcA. Conditional GrgA knock-out leads to significant reduction in RB growth and complete abrogation of progeny EB formation. Based on this, GrgA-mediated transcriptional regulatory network (TRN) was deemed likely to control chlamydial growth, development, and pathogenicity.

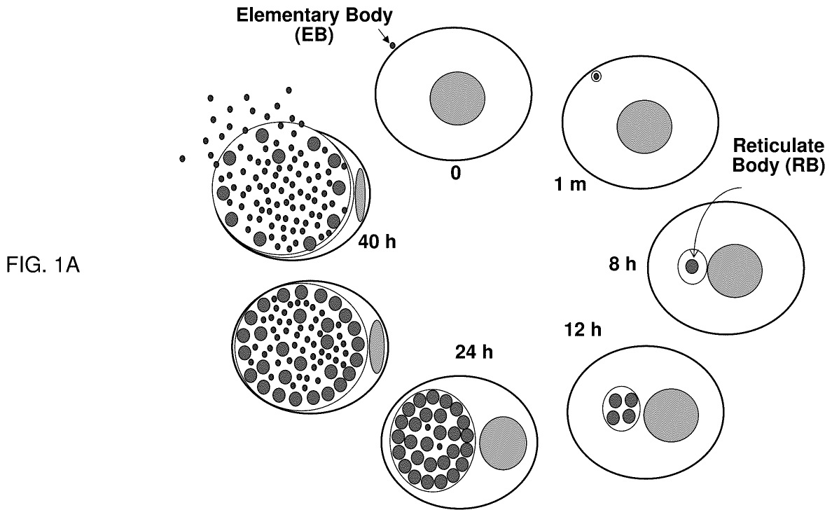

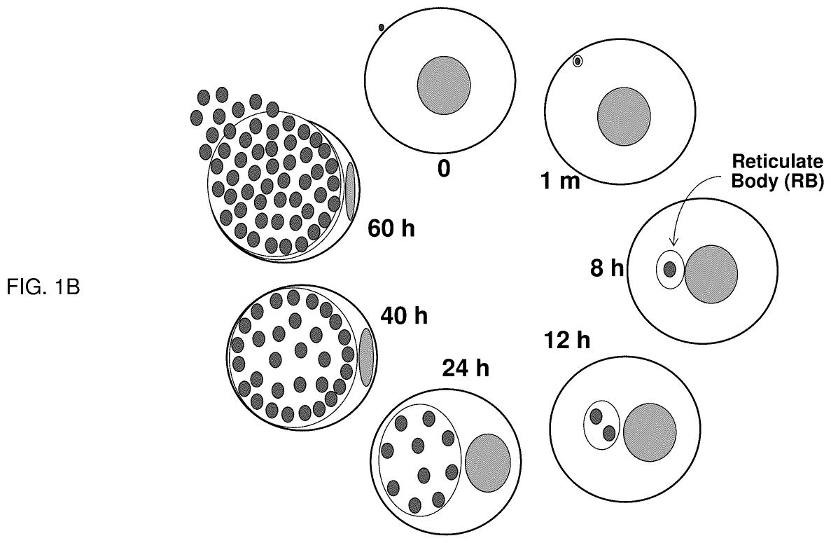

The chlamydial developmental cycle is controlled by the transcriptome, which in turn is controlled by sigma factors of the RNA polymerase and transcription factors. GrgA activates the transcription of multiple chlamydial genes in vitro and in vivo. In this work, a mutant Chlamydia deficient for the grgA gene was developed. This gene was found here to be essential to the conversion of RBs to EBs in the developmental cycle. The mutant Chlamydia forms replicating RBs, but they are unable to differentiate back to infectious EBs. See FIG. 1B. These RBs are still able to elicit an immune response in the host against Chlamydia.

We attempted to disrupt grgA through insertional mutagenesis using a suicidal plasmid carrying a group II intron containing aadA, which confers spectinomycin resistance. However, diagnostic PCR analysis of spectinomycin-resistant Chlamydia from these mutagenesis attempts failed to locate the group II intron within grgA, indicative of off-target insertion. The failure of group II intron to disrupt grgA indicated that grgA is an essential gene. We also attempted to conditionally knock down GrgA expression using CRISPR/dCas9 interference. However, we found that the CRISPR/dCas9 interference system is highly toxic to C. trachomatis, in contrast to results reported in the literature.

### B. GrgA

GrgA is a Chlamydia transcription factor. It is present in both chlamydial cellular forms, the EB and the RB, and stimulates transcription from several σ⁶⁶-dependent promoters and σ²⁸-dependent promoters that are active at different stages. Thus, GrgA can stimulate transcription from several promoters that can control the expression of genes that are critical for chlamydial growth and development.

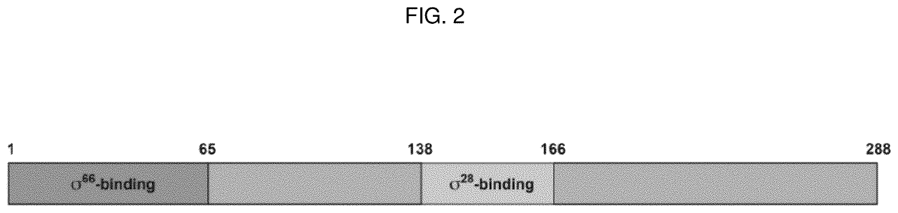

GrgA overexpression has been found to inhibit C. trachomatis growth through σ⁶⁶- and σ²⁸-dependent mechanisms. σ⁶⁶ is the primary sigma factor, necessary for transcription of most chlamydial genes throughout the developmental cycle. See FIG. 2, which shows the domain structure of the wild type GrgA protein.

### C. Chlamydia Compositions

Although the examples presented here focus on Chlamydia trachomatis, the invention is contemplated for use with any chlamydia species and strains within the Chlamydia and Chlamydophila genera. For example, the invention can be used for any strain that is infectious in humans or in animals. Examples include but are not limited to Chlamydia trachomatis, Chlamydia pneumoniae, Chlamydia psittaci, Chlamydia muridarum, Chlamydia suis, Chlamydophila. abortus, Chlamydophila felis, Chlamydophila pecorum, Chlamydia ibidis, Chlamydia avium, Chlamydia gallinacea, and the like.

This composition also optionally includes a carrier such as a suitable medium or buffered solution, and the like, and optional components such as pH adjusters, salts, sugars, and the like.

### D. GrgA Knock-Out

Inventors have discovered that KO of this particular gene has a surprising effect. Finding the right gene is key. The knock-out is a conditional knock-out or gene disruption.

### E. DOPE Technology

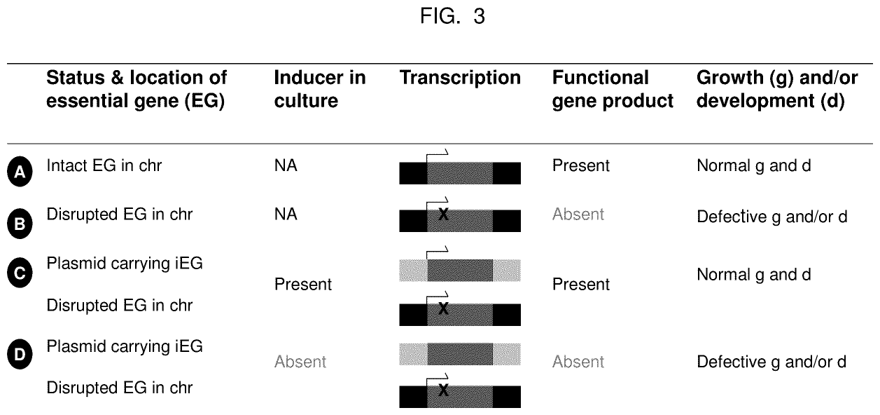

Conceptually, inactivation of the essential genes (e.g., grgA) in Chlamydia using DOPE can be implemented through multiple strategies (e.g., group II intron insertional mutagenesis, homologous recombination, and CRISPR inactivation) in any Chlamydia spp. This methodology is used to determine whether or not a particular gene (any gene) is essential to the developmental cycle of the bacteria. See FIG. 3. First, one determines whether the gene is functional, i.e, whether the cell growth and development is normal when the gene is expressed normally. See FIG. 3, line A. Second, the gene is disrupted and the cells are tested for any failures to develop when the gene is not operational. See FIG. 3, line B. Then, a plasmid is produced to contain the gene, which is inducible, and the plasmid is inserted into the cells. The cells are examined when the gene is induced (FIG. 3, line C) and when the gene is not induced (FIG. 3, line D). If the gene's disruption causes losses in cell development, and this loss is corrected when the gene is induced, then the gene may be considered essential.

The DOPE technology can be used to study essential genes in Chlamydia or in other bacterial species. DOPE offers several advantages over technologies developed for studying essential genes that rely on downregulation using deactivated CRISPR-associate d proteins (dCas) and complementation using constitutive expression from a transformed plasmid. Because specific genes are disrupted by an intron, DOPE is devoid of the “off-target” effects of CRISPR interference. More importantly, DOPE lacks the nonspecific toxicity of dCas9. With a modified, carefully calibrated, and tunable anhydrotetracycline (ATC)-inducible system, DOPE allows for studying essential genes such as GrgA that are toxic when constitutively expressed from a recombinant plasmid.

Derivation of CtL2 with conditional GrgA knock-out (L2/cgad-peig) through DOPE was attempted. The grgA gene was disrupted using type II intron insertional mutagenesis to further investigate the role of GrgA in chlamydial physiology. However, multiple attempts resulted in two transformants whose grgA remained intact. These negative results, together with the failures of saturated chemical mutagenesis studies and transposon mutagenesis to produce grgA-null mutants, suggest the possibility that that grgA is an essential gene. We then attempted to knock down GrgA expression through CRISPR transcription interference using deactivated Cas9 from multiple sources but discovered that the CRISPR/dCas9 systems are toxic in Chlamydia in the absence any guide RNA.

Compared to previously reported plasmid-mediated complementation technologies, DOPE allows for precise expression manipulation of genes of interests and is suitable for studying genes like GrgA whose overexpression is toxic. Unlike CRISPR interference, DOPE does not have off-target effects or general nonspecific toxicity.

### F. Vaccine Compositions

Vaccines and vaccine compositions according to the invention include the conditional GrgA knock-out Chlamydia described herein. The invention relates to a vaccine composition comprising a Chlamydia species with a conditional knock-out of GrgA. Preferably the species is C. trachomatis, however any Chlamydia or Chlamydophis species is suitable. For example, C. pneumoniae, C. psittaci, C. muridarum, C. suis, Chlamydophila. abortus, Chlamydophila felis, Chlamydophila pecorum, C. ibidis, C. avium, C. gallinacea, and the like, also can be used.

The RB of the mutant GrgA knock-out contains MOMP and other proteins that cause effective immunity to the disease. Because of this, the vaccine can produce immunity to these proteins, including the highly conserved antigen, MOMP, similar to natural immunity formed in humans upon exposure to Chlamydia spp. This is an attenuated vaccine, in which the infectivity of the vaccine is preserved, but because it does not form progenies, it is safe to administer.

The vaccine or vaccine composition in general contains some type of carrier for the bacteria, such as an appropriate medium or buffer for administration to a subject. In some preferred embodiments, therefore, the vaccine is administered to a subject as a pharmaceutical composition. This pharmaceutical composition may contain salts, buffers, adjuvants, or other compounds that are desirable for improvement of efficacy. In some embodiments, adjuvants are used in an effort to induce or improve a specific immune response. Descriptions of adjuvants are described in Warren et al. (Ann. Rev. Biochem., 4:369-388, 1986), the entire disclosure of which is hereby incorporated by reference. Examples of materials suitable for use in vaccine compositions are known to those of skill in the art and are described in Remington's Pharmaceutical Sciences (Osol, A, Ed, Mack Publishing Co, Easton, Pa., pp. 1324-1341 (1980), the relevant disclosures of which are incorporated herein by reference).

In some embodiments, the vaccine can be formulated into liquid preparations including aqueous or nonaqueous solutions, suspensions, emulsions, and the like) suitable for injection intravenously, intraarterially, intraperitoneally, or the like, to deliver a systemic administration. Additional components can optionally be included, such as buffer, electrolytes, preservatives, dispersing agents, pH adjusters, osmolality adjusters, sugars, and the like. The vaccine can be provided in a suitable container, such as a vial, a prepared and filled syringe, or any suitable container known in the art, and preferably is sterile.

### G. Methods of Vaccination

The vaccines according to this invention are contemplated to be useful for any animal, including humans, that are susceptible to infection with one or more Chlamydia species. Subjects for vaccination include humans, primates, monkeys, farm animals (including cattle, horses, pigs, sheep, goats, and the like), laboratory animals such as rodents (including mice, rats, guinea pigs, and the like), rabbits and the like, birds (such as chickens, turkeys, geese, ducks, and other poultry), companion animals (including dogs, cats, and the like), and reptiles (including snakes, and the like).

The subjects include any animal that is suffering from a chlamydial infection, suspected of having a chlamydial infection, or potentially susceptible to developing a chlamydial infection. Thus, the vaccine can be used prophylactically or as a treatment. Prophylactic use of the vaccine preferably induces immunity in the subject or host, including neutralizing antibodies, that will reduce the likelihood or severity of chlamydial infection, or preferably prevent the infection. Use as a treatment preferably increases the natural immunity of an infected subject to increase the subject's ability to clear the infection, resulting in faster resolution of the condition.

The administration of the conjugate vaccine (or the antisera which it elicits) may be for either a “prophylactic” or “therapeutic” purpose. When provided prophylactically, the vaccine is provided in advance of any symptom of bacterial infection. The prophylactic administration of the vaccine preferably serves to prevent or attenuate any subsequent infection as discussed above. When provided therapeutically, the vaccine is provided upon the detection of a symptom of actual infection, or a positive test for infection. The therapeutic administration of the vaccine preferably serves to attenuate any actual infection.

### H. Dosing and Dosage Regimens

The particular dosage depends upon the age, weight, sex and medical condition of the subject to be treated, as well as on the method of administration. Suitable doses can be readily determined by those of skill in the art based on these and other factors which are known to the skilled practitioner. One dose or multiple doses may be administered to a single subject.

In preferred embodiments, a suitable amount of vaccine for inducing an immune response in a subject includes administering to a subject in need thereof a therapeutically effective or a prophylactically effective amount. This amount can consist of one dose or a regimen of more than one dose, such as a booster. Therefore, the number of administrations can vary. Administration may be, for example, one time, or administration may be monthly, yearly, or less frequently. The actual amount administered, and the number of doses and boosters given can be determined by the skilled practitioner in the medical arts. This will depend on the age, sex, and weight, of the subject, the stage of the disease, and the severity of what is being treated (including prophylactic treatment). Prescription of treatment, e.g., decisions on dosage is within the responsibility of general practitioners and other medical doctors.

The chlamydiae to be used for vaccines according to this invention are produced using ATC in the culture medium to induce GrgA expression. ATC is removed from the vaccine preparations and is not present in the subject so that when the vaccines are administered to the subject, expression is GrgA is turned off. This prevents continued infection and cause elevated levels of released immunogenic but non-infectious RB.

### I. Conclusion

Since the first publication demonstrating reproducible transformation of Chlamydia with a shuttle vector 12 years ago, the Chlamydia research community has utilized the reverse genetic tool to investigate gene function through ectopic gene overexpression, gene knockdown, and other approaches. Nonetheless, effective strategies for disrupting or depleting truly essential genes have hampered research in Chlamydia and other biological systems. In this report, we developed a tightly regulated inducible expression system termed DOPE that allows for the knockdown of essential genes in Chlamydia. The DOPE system not only represents a convenient and versatile tool for establishing the essentiality of genes, but also defining their underlying mechanisms. Unlike CRISPR interference, DOPE lacks off-target effects or general nonspecific toxicity.

The inability to generate grgA-null mutants by us using gene target mutagenesis and by the Valdivia Lab using random mutagenesis strongly indicated that grgA is crucial for Chlamydia growth and viability. By applying DOPE strategy to knockdown GrgA expression, we show here that GrgA plays a critical role in maintaining RB replication efficiency and is absolutely essential for the RB-to-EB differentiation. Surprisingly, even though previous studies demonstrated that two immediate-early transcriptional factors euo and hrcA are readily upregulated following the induction of GrgA overexpression, genome replication kinetics data presented here suggests that the primary EB-to-RB differentiation is not affected by ATC omission in the culture. However, these data do not exclude the possibility that GrgA plays a role in the primary differentiation because the amount of GrgA prepacked into EBs could be sufficient for supporting the primary EB-to-RB differentiation. In keeping with this view, significant amounts of GrgA were detected in C. trachomatis EBs. Our ongoing transcriptomic analysis will elucidate the mechanisms by which GrgA regulates RB growth and RB-to-EB differentiation.

Formation of EBs is absolutely required for dissemination of chlamydial infection within the infected host and transmission to new hosts. Because RBs and EBs share most of the immunodominant antigens (e.g., major outer membrane protein), conditional GrgA-deficient, “maturation”-defective chlamydiae are potential candidates for life attenuated Chlamydia vaccines, provided that strategies are in place to fully prevent EBs from escaping the gene expression regulatory system in DOPE plasmid. As well, the maturation-defective chlamydiae serve as useful system for studying the roles of RBs in antichlamydial immunity.

In summary, the data presented here strongly suggest that GrgA plays an important role in Chlamydia growth and is essential for RB-to-EB differentiation. The extremely low level of EB formation detected from ATC-free cultures is likely due to leaky GrgA expression in few cells. The conditional GrgA-deficient Chlamydia represents a valuable model for studying RB replication and RB-to-EB differentiation. Maturation-defective chlamydiae, for example, due to GrgA deficiency, can be used as an avirulent vaccine against chlamydial diseases.

### 5. Examples

This invention is not limited to the particular processes, compositions, or methodologies described, as these may vary. The terminology used in the description is for the purpose of describing the particular versions or embodiments only, and is not intended to limit the scope of the present invention which will be limited only by the appended claims. Although any methods and materials similar or equivalent to those described herein can be used in the practice or testing of embodiments of the present invention, the preferred methods, devices, and materials are now described. All publications mentioned herein, are incorporated by reference in their entirety; nothing herein is to be construed as an admission that the invention is not entitled to antedate such disclosure by virtue of prior invention.

### Example 1: General Materials and Methods

### A. Primers and Other Sequences.

The following primers were used here.

TABLE 1
Primers

| Name | Sequence | SEQ ID NO |
|---|---|---|
| Pgp3-pgp4-F | 5′ AGTTCGAATTACGGGGTTT 3′ | 1 |
| His-RBS-R | 5′ GGTAGGTCCTAACTATTCATTT CACTTTTCTC 3′ | 2 |
| RBS-His-F | 5′ ATAGTTAGGACCTACCGGTATG GGCAGCA 3′ | 3 |
| GrgA-67-R | 5′ ATTACGGATACTTAACTTAT ATCCTTCTCTAGATG 3′ | 4 |
| GrgA-67-F | 5′ GTTAAGTATCCGTAATTCGAAA CACTTGTC 3′ | 5 |
| Pgp4-pgp3-R | 5′ ACCCCGTAATTCGAACTTT 3′ | 6 |
| GrgA67_IBS1/2 | 5′ AAAAAAGCTTATAATTATCCTTAATAAGCTATCCA | 7 |
| | GTGCGCCCAGATAGGGTG 3′ | |
| Universal | 5′ CGAAATTAGAAACTTGCGTTCAGTAAAC 3′ | 8 |
| primer | | |
| GrgA67_EBS2 | 5′ TGAACGCAAGTTTCTAATTTCGGTTCTTATO | 9 |
| | CGATAGAGGAAAGTGTCT 3′ | |
| GrgA67_EBS1/ | 5′ CAGATTGTACAAATGTGGTGATAACAGATAAGTC | 10 |
| delta | TATCCATTTAACTTACCTTTCTTTGT 3′ | |
| gA | 5′ ACTGTTATTACATCTAGAGAAGGA 3′ | 11 |
| gB | 5′ AGACATTGGAGCTACAGGTG 3′ | 12 |
| pT | 5′ TAATACGACTCACTATAGGG 3′ | 13 |
| gU | 5′ GAAATAGGCTATGCAACTCG 3 | 14 |

| The sequence of pTargetron-aadA-gagA67 is as follows: | |
|---|---|
| (SEQ ID NO: 15) | |
| 1 | GAATTCCGGATGAGCATTCATCAGGCGGGCAAGAATGTGAATAAAGGCCGGATAAAACTT |
| 61 | GTGCTTATTTTTCTTTACGGTCTTTAAAAAGGCCGTAATATCCAGCTGAACGGTCTGGTT |
| 121 | ATAGGTACATTGAGCAACTGACTGAAATGCCTCAAAATGTTCTTTACGATGCCATTGGGA |
| 181 | TATATCAACGGTGGTATATCCAGTGATTTTTTTCTCCATTTTAGCTTCCTTAGCTCCTGA |
| 241 | AAATCTCGATAACTCAAAAAATACGCCCGGTAGTGATCTTATTTCATTATGGTGAAAGTT |
| 301 | GGAACCTCTTACGTGCCGATCAACGTCTCATTTTCGCCAAAAGTTGGCCCAGGGCTTCCC |
| 361 | GGTATCAACAGGGACACCAGGATTTATTTATTCTGCGAAGTGATCTTCCGTCACAGGTAT |
| 421 | TTATTCGGCGCAAAGTGCGTCGGGTGATGCTGCCAACTTACTGATTTAGTGTATGATGGT |
| 481 | GTTTTTGAGGTGCTCCAGTGGCTTCTGTTTCTATCAGCTGTCCCTCCTGTTCAGCTACTG |
| 541 | ACGGGGTGGTGCGTAACGGCAAAAGCACCGCCGGACATCAGCGCTAGCGGAGTGTATACT |
| 601 | GGCTTACTATGTTGGCACTGATGAGGGTGTCAGTGAAGTGCTTCATGTGGCAGGAGAAAA |
| 661 | AAGGCTGCACCGGTGCGTCAGCAGAATATGTGATACAGGATATATTCCGCTTCCTCGCTC |
| 721 | ACTGACTCGCTACGCTCGGTCGTTCGACTGCGGCGAGCGGAAATGGCTTACGAACGGGGC |
| 781 | GGAGATTTCCTGGAAGATGCCAGGAAGATACTTAACAGGGAAGTGAGAGGGCCGCGGCAA |
| 841 | AGCCGTTTTTCCATAGGCTCCGCCCCCCTGACAAGCATCACGAAATCTGACGCTCAAATC |
| 901 | AGTGGTGGCGAAACCCGACAGGACTATAAAGATACCAGGCGTTTCCCCCTGGCGGCTCCC |
| 961 | TCGTGCGCTCTCCTGTTCCTGCCTTTCGGTTTACCGGTGTCATTCCGCTGTTATGGCCGC |
| 1021 | GTTTGTCTCATTCCACGCCTGACACTCAGTTCCGGGTAGGCAGTTCGCTCCAAGCTGGAC |
| 1081 | TGTATGCACGAACCCCCCGTTCAGTCCGACCGCTGCGCCTTATCCGGTAACTATCGTCTT |
| 1141 | GAGTCCAACCCGGAAAGACATGCAAAAGCACCACTGGCAGCAGCCACTGGTAATTGATTT |
| 1201 | AGAGGAGTTAGTCTTGAAGTCATGCGCCGGTTAAGGCTAAACTGAAAGGACAAGTTTTGG |
| 1261 | TGACTGCGCTCCTCCAAGCCAGTTACCTCGGTTCAAAGAGTTGGTAGCTCAGAGAACCTT |
| 1321 | CGAAAAACCGCCCTGCAAGGCGGTTTTTTCGTTTTCAGAGCAAGAGATTACGCGCAGACC |
| 1381 | AAAACGATCTCAAGAAGATCATCTTATTAATCAGATAAAATATTTCTAGCTAGATTTCAG |
| 1441 | TGCAATTTATCTCTTCAAATGTAGCACCTGAAGTCAGCCCCATACGATATAAGTTGTAAT |
| 1501 | TCTCATGTTTGACAGCTTATCATCGATAAGCTCAAGGAGATGGCGCCCAACAGTCCCCCG |
| 1561 | GCCACGGGGCCTGCCACCATACCCACGCCGAAACAAGCGCTCATGAGCCCGAAGTGGCGA |
| 1621 | GCCCGATCTTCCCCATCGGTGATGTCGGCGATATAGGCGCCAGCAACCGCACCTGTGGCG |
| 1681 | CCGGTGATGCCGGCCACGATGCGTCCGGCGTAGAGGATCGAGATCTCGATCCCGCGAAAT |
| 1741 | TAATACGACTCACTATAGGGGAATTGTGAGCGGATAACAATTCCCCTCTAGAGGTACCGG |
| 1801 | ATCCTCCAAATTATTCCTTACATGAATTTTTTGTCTGAGCGACTTTCTCCCATTGAAAAA |
| 1861 | GATTTTCTTAAACAAAACGTGCTTTACTTCTTGCAGAAAAATCGGTAAACTTGCCGTTTC |
| 1921 | GTCTAGGCAGACTCGTCCGCGTCTTTTTTCAAAACTCCCTTTTTAGGAAGTTTTTGAAGG |
| 1981 | CGTTCCTCAGATTTTCCCGAGTTGGAGGAGACTGGCCGGCACTACAAGCTCATATCAAGG |
| 2041 | TAAGGAAAGATTTCCAAGCTTATAATTATCCTTAATAAGCTATCCAGTGCGCCCAGATAG |
| 2101 | GGTGTTAAGTCAAGTAGTTTAAGGTACTACTCTGTAAGATAACACAGAAAACAGCCAACC |
| 2161 | TAACCGAAAAGCGAAAGCTGATACGGGAACAGAGCACGGTTGGAAAGCGATGAGTTACCT |
| 2221 | AAAGACAATCGGGTACGACTGAGTCGCAATGTTAATCAGATATAAGGTATAAGTTGTGTT |
| 2281 | TACTGAACGCAAGTTTCTAATTTCGGTTCTTATCCGATAGAGGAAAGTGTCTGAAACCTC |
| 2341 | TAGTACAAAGAAAGGTAAGTTAAATGGATAGACTTATCTGTTATCACCACATTTGTACAA |
| 2401 | TCTGTAGGAGAACCTATGGGAACGAAACGAAAGCGATGCCGAGAATCTGAATTTACCAAG |
| 2461 | ACTTAACACTAACTGGGGATACCCTAAACAAGAATGCCTAATAGAAAGGAGGAAAAAGGC |
| 2521 | TATAGCACTAGAGCTTGAAAATCTTGCAAGGGTACGGAGTACTCGTAGTAGTCTGAGAAG |
| 2581 | GGTAACGCCCTTTACATGGCAAAGGGGTACAGTTATTGTGTACTAAAATTAAAAATTGAT |
| 2641 | TAGGGAGGAAAACCTCAAAATGAAACCAACAATGGCAATTTTAGAAAGAATCAGTAAAAA |
| 2701 | TTCACAAGAAAATATAGACGAAGTTTTTACAAGACTTTATCGTTATCTTTTACGTCCAGA |
| 2761 | TATTTATTACGTGGCGACGCGTTGCCTGACGATGCGTGGAGACCGAAACCTTGCGCTCGT |
| 2821 | TCGCCAGCCAGGACAGAAATGCCTCGACTTCGCTGCTGCCCAAGGTTGCCGGGTGACGCA |
| 2881 | CACCGTGGAAACGGATGAAGGCACGAACCCAGTGGACATAAGCCTGTTCGGTTCGTAAGC |
| 2941 | TGTAATGCAAGTAGCGTATGCGCTCACGCAACTGGTCCAGAACCTTGACCGAACGCAGCG |
| 3001 | GTGGTAACGGCGCAGTGGCGGTTTTCATGGCTTGTTATGACTGTTTTTTTGGGGTACAGT |
| 3061 | CTATGCCTCGGGCATCCAAGCAGCAAGCGCGTTACGCCGTGGGTCGATGTTTGATGTTAT |
| 3121 | GGAGCAGCAACGATGTTACGCAGCAGGGCAGTCGCCCTAAAACAAAGTTAAACATCATGA |
| 3181 | GGGAAGCGGTGATCGCCGAAGTATCGACTCAACTATCAGAGGTAGTTGGCGTCATCGAGC |
| 3241 | GCCATCTCGAACCGACGTTGCTGGCCGTACATTTGTACGGCTCCGCAGTGGATGGCGGCC |
| 3301 | TGAAGCCACACAGTGATATTGATTTGCTGGTTACGGTGACCGTAAGGCTTGATGAAACAA |
| 3361 | CGCGGCGAGCTTTGATCAACGACCTTTTGGAAACTTCGGCTTCCCCTGGAGAGAGCGAGA |
| 3421 | TTCTCCGCGCTGTAGAAGTCACCATTGTTGTGCACGACGACATCATTCCGTGGCGTTATC |
| 3481 | CAGCTAAGCGCGAACTGCAATTTGGAGAATGGCAGCGCAATGACATTCTTGCAGGTATCT |
| 3541 | TCGAGCCAGCCACGATCGACATTGATCTGGCTATCTTGCTGACAAAAGCAAGAGAACATA |
| 3601 | GCGTTGCCTTGGTAGGTCCAGCGGCGGAGGAACTCTTTGATCCGGTTCCTGAACAGGATC |
| 3661 | TATTTGAGGCGCTAAATGAAACCTTAACGCTATGGAACTCGCCGCCCGACTGGGCTGGCG |
| 3721 | ATGAGCGAAATGTAGTGCTTACGTTGTCCCGCATTTGGTACAGCGCAGTAACCGGCAAAA |
| 3781 | TCGCGCCGAAGGATGTCGCTGCCGACTGGGCAATGGAGCGCCTGCCGGCCCAGTATCAGC |
| 3841 | CCGTCATACTTGAAGCTAGACAGGCTTATCTTGGACAAGAAGAAGATCGCTTGGCCTCGC |
| 3901 | GCGCAGATCAGTTGGAAGAATTTGTCCACTACGTGAAAGGCGAGATCACCAAGGTAGTCG |
| 3961 | GCAAATAATGTCTAACAATTCGTTCAAGCCGACGCCGCTTCGCGGCGCGGCTTAACTCAA |
| 4021 | GCGTTAGATGCACTAAGCACATAATTGCTCACAGCCAAACTATCAGGCCCGGGACGCGTT |
| 4081 | GGGAAATGGCAATGATAGCGAAACAACGTAAAACTCTTGTTGTATGCTTTCATTGTCATC |
| 4141 | GTCACGTGATTCATAAACACAAGTGAATGTCGACAGTGAATTTTTACGAACGAACAATAA |
| 4201 | CAGAGCCGTATACTCCGAGAGGGGTACGTACGGTTCCCGAAGAGGGTGGTGCAAACCAGT |
| 4261 | CACAGTAATGTGAACAAGGCGGTACCTCCCTACTTCACCATATCATTTTCTGCAGCCCCC |
| 4321 | TAGAAATAATTTTGTTTAACTTTAAGAAGGAGATATACATATATGGCTAGATCGTCCATT |
| 4381 | CCGACAGCATCGCCAGTCACTATGGCGTGCTGCTAGCGCTATATGCGTTGATGCAATTTC |
| 4441 | TATGCACTCGTAGTAGTCTGAGAAGGGTAACGCCCTTTACATGGCAAAGGGGTACAGTTA |
| 4501 | TTGTGTACTAAAATTAAAAATTGATTAGGGAGGAAAACCTCAAAATGAAACCAACAATGG |
| 4561 | CAATTTTAGAAAGAATCAGTAAAAATTCACAAGAAAATATAGACGAAGTTTTTACAAGAC |
| 4621 | TTTATCGTTATCTTTTACGTCCAGATATTTATTACGTGGCGTATCAAAATTTATATTCCA |
| 4681 | ATAAAGGAGCTTCCACAAAAGGAATATTAGATGATACAGCGGATGGCTTTAGTGAAGAAA |
| 4741 | AAATAAAAAAGATTATTCAATCTTTAAAAGACGGAACTTACTATCCTCAACCTGTACGAA |
| 4801 | GAATGTATATTGCAAAAAAGAATTCTAAAAAGATGAGACCTTTAGGAATTCCAACTTTCA |
| 4861 | CAGATAAATTGATCCAAGAAGCTGTGAGAATAATTCTTGAATCTATCTATGAACCGGTAT |
| 4921 | TCGAAGATGTGTCTCACGGTTTTAGACCTCAACGAAGCTGTCACACAGCTTTGAAAACAA |
| 4981 | TCAAAAGAGAGTTTGGCGGCGCAAGATGGTTTGTGGAGGGAGATATAAAAGGCTGCTTCG |
| 5041 | ATAATATAGACCACGTTACACTCATTGGACTCATCAATCTTAAAATCAAAGATATGAAAA |
| 5101 | TGAGCCAATTGATTTATAAATTTCTAAAAGCAGGTTATCTGGAAAACTGGCAGTATCACA |
| 5161 | AAACTTACAGCGGAACACCTCAAGGTGGAATTCTATCTCCTCTTTTGGCCAACATCTATC |
| 5221 | TTCATGAATTGGATAAGTTTGTTTTACAACTCAAAATGAAGTTTGACCGAGAAAGTCCAG |
| 5281 | AAAGAATAACACCTGAATATCGGGAGCTCCACAATGAGATAAAAAGAATTTCTCACCGTC |
| 5341 | TCAAGAAGTTGGAGGGTGAAGAAAAAGCTAAAGTTCTTTTAGAATATCAAGAAAAACGTA |
| 5401 | AAAGATTACCCACACTCCCCTGTACCTCACAGACAAATAAAGTATTGAAATACGTCCGGT |
| 5461 | ATGCGGACGACTTCATTATCTCTGTTAAAGGAAGCAAAGAGGACTGTCAATGGATAAAAG |
| 5521 | AACAATTAAAACTTTTTATTCATAACAAGCTAAAAATGGAATTGAGTGAAGAAAAAACAC |
| 5581 | TCATCACACATAGCAGTCAACCCGCTCGTTTTCTGGGATATGATATACGAGTAAGGAGAT |
| 5641 | CTGGAACGATAAAACGATCTGGTAAAGTCAAAAAGAGAACACTCAATGGGAGTGTAGAAC |
| 5701 | TCCTTATTCCTCTTCAAGACAAAATTCGTCAATTTATTTTTGACAAGAAAATAGCTATCC |
| 5761 | AAAAGAAAGATAGCTCATGGTTTCCAGTTCACAGGAAATATCTTATTCGTTCAACAGACT |
| 5821 | TAGAAATCATCACAATTTATAATTCTGAACTCCGCGGGATTTGTAATTACTACGGTCTAG |
| 5881 | CAAGTAATTTTAACCAGCTCAATTATTTTGCTTATCTTATGGAATACAGCTGTCTAAAAA |
| 5941 | CGATAGCCTCCAAACATAAGGGAACACTTTCAAAAACCATTTCCATGTTTAAAGATGGAA |
| 6001 | GTGGTTCGTGGGGGATCCCGTATGAGATAAAGCAAGGTAAGCAGCGCCGTTATTTTGCAA |
| 6061 | ATTTTAGTGAATGTAAATCCCCTTATCAATTTACGGATGAGATAAGTCAAGCTCCTGTAT |
| 6121 | TGTATGGCTATGCCCGGAATACTCTTGAAAACAGGTTAAAAGCTAAATGTTGTGAATTAT |
| 6181 | GTGGGACGTCTGATGAAAATACTTCCTATGAAATTCACCATGTCAATAAGGTCAAAAATC |
| 6241 | TTAAAGGCAAAGAAAAATGGGAAATGGCAATGATAGCGAAACAACGTAAAACTCTTGTTG |
| 6301 | TATGCTTTCATTGTCATCGTCACGTGATTCATAAACACAAGTGAATGTCGAGCACCCGTT |
| 6361 | CTCGGAGCACTGTCCGACCGCTTTGGCCGCCGCCCAGTCCTGCTCGCTTCGCTACTTGGA |
| 6421 | GCCACTATCGACTACGCGATCATGGCGACCACACCCGTCCTGTGGATCGCCAAGCTCGCC |
| 6481 | GATGGTAGTGTGGGGTCTCCCCATGCGAGAGTAGGGAACTGCCAGGCATCAAATAAAACG |
| 6541 | AAAGGCTCAGTCGAAAGACTGGGCCTTTCGTTTTATCTGTTGTTTGTCGGTGAACGCTCT |
| 6601 | CCTGAGTAGGACAAATCCGCCGGGAGCGGATTTGAACGTTGCGAAGCAACGGCCCGGAGG |
| 6661 | GTGGCGGGCAGGACGCCCGCCATAAACTGCCAGGCATCAAATTAAGCAGAAGGCCATCCT |
| 6721 | GACGGATGGCCTTTTTGCGTTTCTACAAACTCTTCCTGTCGTCATATCTACAAGCCATCC |
| 6781 | CCCCACAGATACGGTAAACTAGCCTCGTTTTTGCATCAGGAAAGCAGAACGCCATGAGCG |
| 6841 | GCCTCATTTCTTATTCTGAGTTACAACAGTCCGCACCGCTGTCCGGTAGCTCCTTCCGGT |
| 6901 | GGGCGCGGGGCATGACTATCGTCGCCGCACTTATGACTGTCTTCTTTATCATGCAACTCG |
| 6961 | TAGGACAGGTGCCGGCAGCGCCCAACAGTCCCCCGGCCACGGGGCCTGCCACCATACCCA |
| 7021 | CGCCGAAACAAGCGCCCTGCACCATTATGTTCCGGATCTGCATCGCAGGATGCTGCTGGC |
| 7081 | TACCCTGTGGAACACCTACATCTGTATTAACGAAGCGCTAACCGTTTTTATCAGGCTCTG |
| 7141 | GGAGGCAGAATAAATGATCATATCGTCAATTATTACCTCCACGGGGAGAGCCTGAGCAAA |
| 7201 | CTGGCCTCAGGCATTTGAGAAGCACACGGTCACACTGCTTCCGGTAGTCAATAAACCGGT |
| 7261 | AAACCAGCAATAGACATAAGCGGCTATTTAACGACCCTGCCCTGAACCGACGACCGGGTC |
| 7321 | GAATTTGCTTTCGAATTTCTGCCATTCATCCGCTTATTATCACTTATTCAGGCGTAGCAC |
| 7381 | CAGGCGTTTAAGGGCACCAATAACTGCCTTAAAAAAATTACGCCCCGCCCTGCCACTCAT |
| 7441 | CGCAGTACTGTTGTAATTCATTAAGCATTCTGCCGACATGGAAGCCATCACAGACGGCAT |
| 7501 | GATGAACCTGAATCGCCAGCGGCATCAGCACCTTGTCGCCTTGCGTATAATATTTGCCCA |
| 7561 | TGGTGAAAACGGGGGCGAAGAAGTTGTCCATATTGGCCACGTTTAAATCAAAACTGGTGA |
| 7621 | AACTCACCCAGGGATTGGCTGAGACGAAAAACATATTCTCAATAAACCCTTTAGGGAAAT |
| 7681 | AGGCCAGGTTTTCACCGTAACACGCCACATCTTGCGAATATATGTGTAGAAACTGCCGGA |
| 7741 | AATCGTCGTGGTATTCACTCCAGAGCGATGAAAACGTTTCAGTTTGCTCATGGAAAACGG |
| 7801 | TGTAACAAGGGTGAACACTATCCCATATCACCAGCTCACCGTCTTTCATTGCCATACG |
| The sequence of pTRL2-peig is as follows: | |
| (SEQ ID NO: 16) | |
| 1 | ATGGAAAATAGAGGTACCATGGTCTCTGAACTTATCAAAGAAAATATGCACATGAAATTA |
| 61 | TACATGGAAGGCACTGTCAACAATCATCACTTTAAATGCACCTCTGAAGGTGAAGGCAAA |
| 121 | CCGTATGAAGGAACTCAAACAATGCGCATTAAAGCTGTAGAAGGAGGTCCTCTTCCGTTT |
| 181 | GCTTTCGATATCCTGGCAACTTCTTTCATGTACGGTTCTAAAACCTTCATCAATCATACG |
| 241 | CAAGGCATCCCTGATTTCTTTAAACAGTCTTTTCCGGAAGGCTTCACTTGGGAACGCGTA |
| 301 | ACTACATATGAAGATGGCGGGGTCCTGACCGCGACGCAAGATACATCTCTGCAGGATGGA |
| 361 | TGTCTTATCTACAACGTTAAAATCCGTGGGGTGAATTTTCCATCTAACGGACCTGTTATG |
| 421 | CAAAAGAAAACTCTGGGGTGGGAAGCGTCTACTGAAACATTATATCCAGCCGATGGAGGT |
| 481 | CTTGAAGGTCGTGCGGATATGGCCCTGAAATTAGTGGGGGGGGGACACCTTATTTGTAAT |
| 541 | CTGAAAACCACGTATCGCTCTAAAAAACCGGCTAAAAACCTGAAAATGCCAGGTGTATAT |
| 601 | TACGTCGATCGTCGCTTAGAACGTATCAAAGAAGCAGATAAAGAAACTTACGTTGAACAG |
| 661 | CATGAAGTTGCTGTGGCACGTTACTGCGATTTACCTTCTAAACTTGGACACCGCTAAGGC |
| 721 | GCCAGGGTTCCGATTTAGTGCTTTACGGCACCTCGACCCCAAAAAACTTGATTAGGGTGA |
| 781 | TGGTTCACGTAGTGGGCCATCGCCCTGATAGACGGTTTTTCGCCCTTTGACGTTGGAGTC |
| 841 | CACGTTCTTTAATAGTGGACTCTTGTTCCAAACTGGAACAACACTCAACCCTATCTCGGT |
| 901 | CTATTCTTTTGATTTATAAGGGATTTTGCCGATTTCGGCCTATTGGTTAAAAAATGAGCT |
| 961 | GATTTAACAAAAATTTAACGCGAATTTTAACAAAATATTAACGCTTACAATTTCAGGTGG |
| 1021 | CACTTTTCGGGGAAATGTGCGCGGAACCCCTATTTGTTTATTTTTCTAAATACATTCAAA |
| 1081 | TATGTATCCGCTCATGAGACAATAACCCTGATAAATGCTTCAATAATATTGAAAAAGGAA |
| 1141 | GAGTATGAGTATTCAACATTTCCGTGTCGCCCTTATTCCCTTTTTTGCGGCATTTTGCCT |
| 1201 | TCCTGTTTTTGCTCACCCAGAAACGCTGGTGAAAGTAAAAGATGCTGAAGATCAGTTGGG |
| 1261 | TGCACGAGTGGGTTACATCGAACTGGATCTCAACAGCGGTAAGATCCTTGAGAGTTTTCG |
| 1321 | CCCCGAAGAACGTTTTCCAATGATGAGCACTTTTAAAGTTCTGCTATGTGGCGCGGTATT |
| 1381 | ATCCCGTATTGACGCCGGGCAAGAGCAACTCGGTCGCCGCATACACTATTCTCAGAATGA |
| 1441 | CTTGGTTGAGTACTCACCAGTCACAGAAAAGCATCTTACGGATGGCATGACAGTAAGAGA |
| 1501 | ATTATGCAGTGCTGCCATAACCATGAGTGATAACACTGCGGCCAACTTACTTCTGACAAC |
| 1561 | GATCGGAGGACCGAAGGAGCTAACCGCTTTTTTGCACAACATGGGGGATCATGTAACTCG |
| 1621 | CCTTGATCGTTGGGAACCGGAGCTGAATGAAGCCATACCAAACGACGAGCGTGACACCAC |
| 1681 | GATGCCTGTAGCAATGGCAACAACGTTGCGCAAACTATTAACTGGCGAACTACTTACTCT |
| 1741 | AGCTTCCCGGCAACAATTGATAGACTGGATGGAGGCGGATAAAGTTGCAGGACCACTTCT |
| 1801 | GCGCTCGGCCCTTCCGGCTGGCTGGTTTATTGCTGATAAATCTGGAGCCGGTGAGCGTGG |
| 1861 | CTCTCGCGGTATCATTGCAGCACTGGGGCCAGATGGTAAGCCCTCCCGTATCGTAGTTAT |
| 1921 | CTACACGACGGGGAGTCAGGCAACTATGGATGAACGAAATAGACAGATCGCTGAGATAGG |
| 1981 | TGCCTCACTGATTAAGCATTGGTAGGAATTAATGATGTCTCGTTTAGATAAAAGTAAAGT |
| 2041 | GATTAACAGCGCATTAGAGCTGCTTAATGAGGTCGGAATCGAAGGTTTAACAACCCGTAA |
| 2101 | ACTCGCCCAGAAGCTAGGTGTAGAGCAGCCTACATTGTATTGGCATGTAAAAAATAAGCG |
| 2161 | GGCTTTGCTCGACGCCTTAGCCATTGAGATGTTAGATAGGCACCATACTCACTTTTGCCC |
| 2221 | TTTAGAAGGGGAAAGCTGGCAAGATTTTTTACGTAATAACGCTAAAAGTTTTAGATGTGC |
| 2281 | TTTACTAAGTCATCGCGATGGAGCAAAAGTACATTTAGGTACACGGCCTACAGAAAAACA |
| 2341 | GTATGAAACTCTCGAAAATCAATTAGCCTTTTTATGCCAACAAGGTTTTTCACTAGAGAA |
| 2401 | TGCATTATATGCACTCAGCGCAGTGGGGCATTTTACTTTAGGTTGCGTATTGGAAGATCA |
| 2461 | AGAGCATCAAGTCGCTAAAGAAGAAAGGGAAACACCTACTACTGATAGTATGCCGCCATT |
| 2521 | ATTACGACAAGCTATCGAATTATTTGATCACCAAGGTGCAGAGCCAGCCTTCTTATTCGG |
| 2581 | CCTTGAATTGATCATATGCG ATTAGAAAAACAACTTAAATGTGAAAGTGGGTCTTAAAA |
| 2641 | GCAGCATAACCTTTTTCCGTGATGGTAACTTCACTAGTTTAAAAGGATCTAGGTGAAGAT |
| 2701 | CCTTTTTGATAATCTCATGACCAAAATCCCTTAACGTGAGTTTTCGTTCCACTGAGCGTC |
| 2761 | AGACCCCGTAGAAAAGATCAAAGGATCTTCTTGAGATCCTTTTTTTCTGCGCGTAATCTG |
| 2821 | CTGCTTGCAAACAAAAAAACCACCGCTACCAGCGGTGGTTTGTTTGCCGGATCAAGAGCT |
| 2881 | ACCAACTCTTTTTCCGAAGGTAACTGGCTTCAGCAGAGCGCAGATACCAAATACTGTCCT |
| 2941 | TCTAGTGTAGCCGTAGTTAGGCCACCACTTCAAGAACTCTGTAGCACCGCCTACATACCT |
| 3001 | CGCTCTGCTAATCCTGTTACCAGTGGCTGCTGCCAGTGGCGATAAGTCGTGTCTTACCGG |
| 3061 | GTTGGACTCAAGACGATAGTTACCGGATAAGGCGCAGCGGTCGGGCTGAACGGGGGGTTC |
| 3121 | GTGCACACAGCCCAGCTTGGAGCGAACGACCTACACCGAACTGAGATACCTACAGCGTGA |
| 3181 | GCTATGAGAAAGCGCCACGCTTCCCGAAGGGAGAAAGGCGGACAGGTATCCGGTAAGCGG |
| 3241 | CAGGGTCGGAACAGGAGAGCGCACGAGGGAGCTTCCAGGGGGAAACGCCTGGTATCTTTA |
| 3301 | TAGTCCTGTCGGGTTTCGCCACCTCTGACTTGAGCGTCGATTTTTGTGATGCTCGTCAGG |
| 3361 | GGGGCGGAGCCTATGGAAAAACGCCAGCAACGCGGCCTTTTTACGGTTCCTGGCCTTTTG |
| 3421 | CTGGCCTTTTGCTCACATGACCCGACACCATCGAATGGCCAGATGATTAATTCCTAATTT |
| 3481 | TTGTTGACACTCTATCATTGATAGAGTTATTTTACCACTCCCTATCAGTGATAGAGAAAA |
| 3541 | GTGAAATGAATAGTTAGGACCTACCGGTATGGGCAGCAGCCATCATCATCATCATCACAG |
| 3601 | CAGCGGCCTGGTGCCGCGCGGCAGCCATATGTATTTTACAAGAGATCCAGTCATAGAGAC |
| 3661 | TGTTATTACATCTAGAGAAGGATATAAGTTAAGTATCCGTAATTCGAAACACTTGTCCCA |
| 3721 | AGATCCTTTTGTCGTTGAGGCTATAGAGGTTGTCCGTTTAGGAGGGACTAGTTTTTTCCG |
| 3781 | TAATTGTGATCATAGTAAGCCGTTTTTACTGCCAGCATCTGATTATGAAGTGATGGAAAT |
| 3841 | CCGGGATGCTAAAATCAACCTTAAAGCTGTTGGTTTAGATCGTGGAGTCAAGATTGTTGG |
| 3901 | TAGTCGGGAAGCTTTACTAAAGATGCCGAAGGTGGCTCCAATAGTTTCTGTATCGGAAGA |
| 3961 | TAATACGATTGTTTCTGAAGAAGAGGTAGTTGCAGACTCTACTGTTGCAGCTCCCGCTTC |
| 4021 | TACACCTGTAGCTCCAATGTCTAAGAAAGAGAGACGAAAAGAGTTTAAGAATGAGAAATG |
| 4081 | GAAGGATAAGAAAAAACAAGGACGTCGTCGAAATAGTAAAGAGATTGCCGATGCTGTTGG |
| 4141 | ATCTTCTCAAGAGATGATCGACACCGTAGCAGAGGAATGTTTGCAAGAGTCCTCTTCTGA |
| 4201 | GGAAGGCGATTTCAGTGAGCGACGGTTTTCTTTGATTCCTCCTCCTACTCGATTGATTTC |
| 4261 | TGATGGTCCAGAAGAACCCGAGGAAGAGTCTCAGCCTGTGACTTCAGTGGATTTAAATGA |
| 4321 | GTCTCTAAACGCTTTAGTCAGCGAAAGTTGCAATGTTATTGAGTCTATTTTAGCCGATGA |
| 4381 | GGACACGGTTGTTTTTACTAAAGAAAAAGATCAAACTGCTGAAGAATCTCAAGAGCAGCC |
| 4441 | AAGTCTTTCATTAGAAGAAACTCCTGTTCATGACAGTATTTCTTCAGAAGAGTAAAGTGA |
| 4501 | AAAATGGCGCACATTGTGCGACATTTTTTTTGTCTGCCGTTTACCGCTACTGCGTCACGG |
| 4561 | ATCTCCACGCGCCCTGTAGCGGCGCATTAAGCGCGGCGGGTGTGGTGGTTACGCGCAGCG |
| 4621 | TGACCGCTACACTTGCCAGCGCCCTAGCGCCCGCTCCTTTCGCTTTCTTCCCTTCCTTTC |
| 4681 | TCGCCACGTTCGCCGGCTTTCCCCGTCAAGCTCTAAAGGGCCCAAGAGGTAAGTCCTCTA |
| 4741 | GTACAAACACCCCCAATATTGTGATATAATTAAAATTATATTCATATTCTGTTGCCAGAA |
| 4801 | AAAACACTTTTAGGCTATATTAGAGCCATCTTCTTTGAAGCGTTGTCTTCTCGAGAAGAT |
| 4861 | TTATCGTACGCAAATATCATCTTTGCGGTTGCGTGTCCTGTGACCTTCATTATGTCGGAG |
| 4921 | TCTGAGCACCCTAGGCGTTTGTACTCCGTCACAGCGGTTGCTCGAAGCACGTGCGGGGTT |
| 4981 | ATCTTAAAAGGGATTGCAGCTTGTAGTCCTGCTTGAGAGAACGTGCGGGCGATTTGCCTT |
| 5041 | AACCCCACCATTTTTCCGGAGCGAGTTACGAAGACAAAACCTCTTCGTTGACCGATGTAC |
| 5101 | TCTTGTAGAAAGTGCATAAACTTCTGAGGATAAGTTATAATAATCCTCTTTTCTGTCTGA |
| 5161 | CGGTTCTTAAGCTGGGAGAAAGAAATGGTAGCTTGTTGGAAACAAATCTGACTAATCTCC |
| 5221 | AAGCTTAAGACTTCAGAGGAGCGTTTACCTCCTTGGAGCATTGTCTGGGCGATCAACCAA |
| 5281 | TCCCGGGCATTGATTTTTTTTAGCTCTTTTAGGAAGGACGCTGTTTGCAAACTGTTCATC |
| 5341 | GCATCTGTTTTTACTATTTCCCTGGTTTTAAAAAATGTTCGACTATTTTCTTGTTTAGAA |
| 5401 | GGTTGCGCTATAGCGACTATTCCTTGAGTCATCCTGTTTAGGAATCTTGTTAAGGAAATA |
| 5461 | TAGCTTGCTGCTCGAACTTGTTTAGTACCTTCGGTCCAAGAAGTCTTGGCAGAGGAAACT |
| 5521 | TTTTTAATCGCATCTAGAATTAGATTATGATTTAAAAGGGAAAACTCTTGCAGATTCATA |
| 5581 | TCCAAGGACAATAGACCAATCTTTTCTAAAGACAAAAAAGATCCTCGATATGATCTACAA |
| 5641 | GTATGTTTGTTGAGTGATGCGGTCCAATGCATAATAACTTCGAATAAGGAGAAGCTTTTC |
| 5701 | ATGCGTTTCCAATAGGATTCTTGGCGAATTTTTAAAACTTCCTGATAAGACTTTTCGCTA |
| 5761 | TATTCTAACGACATTTCTTGCTGCAAAGATAAAATCCCTTTACCCATGAAATCCCTCGTG |
| 5821 | ATATAACCTATCCGTAAAATGTCCTGATTAGTGAAATAATCAGGTTGTTAACAGGATAGC |
| 5881 | ACGCTCGGTATTTTTTTATATAAACATGAAAACTCGTTCCGAAATAGAAAATCGCATGCA |
| 5941 | AGATATCGAGTATGCGTTGTTAGGTAAAGCTCTGATATTTGAAGACTCTACTGAGTATAT |
| 6001 | TCTGAGGCAGCTTGCTAATTATGAGTTTAAGTGTTCTCATCATAAAAACATATTCATAGT |
| 6061 | ATTTAAATACTTAAAAGACAATGGATTACCTATAACTGTAGACTCGGCTTGGGAAGAGCT |
| 6121 | TTTGCGGCGTCGTATCAAAGATATGGACAAATCGTATCTCGGGTTAATGTTGCATGATGC |
| 6181 | TTTATCAAATGACAAGCTTAGATCCGTTTCTCATACGGTTTTCCTCGATGATTTGAGCGT |
| 6241 | GTGTAGCGCTGAAGAAAATTTGAGTAATTTCATTTTCCGCTCGTTTAATGAGTACAATGA |
| 6301 | AAATCCATTGCGTAGATCTCCGTTTCTATTGCTTGAGCGTATAAAGGGAAGGCTTGACAG |
| 6361 | TGCTATAGCAAAGACTTTTTCTATTCGCAGCGCTAGAGGCCGGTCTATTTATGATATATT |
| 6421 | CTCACAGTCAGAAATTGGAGTGCTGGCTCGTATAAAAAAAAGACGAGCAACGTTCTCTGA |
| 6481 | GAATCAAAATTCTTTCTTTGATGCCTTCCCAACAGGATACAAGGATATTGATGATAAAGG |
| 6541 | AGTTATCTTAGCTAAAGGTAATTTCGTGATTATAGCAGCTAGGCCATCTATAGGGAAAAC |
| 6601 | TGCTTTAGCTATAGACATGGCGATAAATCTTGCGGTTACTCAACAGCGTAGAGTTGGTTT |
| 6661 | CCTATCTCTAGAAATGAGCGCAGGTCAAATTGTTGAGCGGATTATTGCTAATTTAACAGG |
| 6721 | AATATCTGGTGAAAAATTACAAAGAGGGGATCTCTCTAAAGAAGAATTATTCCGAGTAGA |
| 6781 | AGAAGCTGGAGAAACAGTTAGAGAATCACATTTTTATATCTGCAGTGATAGTCAGTATAA |
| 6841 | GCTTAATTTAATCGCGAATCAGATCCGGTTGCTGAGAAAGGAAGATCGAGTAGACGTAAT |
| 6901 | ATTTATCGATTACTTGCAGTTGATCAACTCATCGGTTGGAGAAAATCGTCAAAATGAAAT |
| 6961 | AGCAGATATATCTAGAACCTTAAGAGGTTTAGCCTCAGAGCTAAACATTCCTATAGTTTG |
| 7021 | TTTATCCCAACTATCTAGAAAAGTTGAGGATAGAGCAAATAAAGTTCCCATGCTTTCAGA |
| 7081 | TTTGCGAGACAGCGGTCAAATAGAGCAAGACGCAGATGTGATTTTGTTTATCAATAGGAA |
| 7141 | GGAATCGTCTTCTAATTGTGAGATAACTGTTGGGAAAAATAGACATGGATCGGTTTTCTC |
| 7201 | TTCGGTATTACATTTCGATCCAAAAATTAGTAAATTCTCCGCTATTAAAAAAGTATGGTA |
| 7261 | AATTATAGTAACTGCCACTTCATCAAAAGTCCTATCCACCTTGAAAATCAGAAGTTTGGA |
| 7321 | AGAAGACCTGGTCAATCTATTAAGATATCTCCCAAATTGGCTCAAAATGGGATGGTAGAA |
| 7381 | GTTATAGGTCTTGATTTTCTTTCATCTCATTACCATGCATTAGCAGCTATCCAAAGATTG |
| 7441 | CTGACTGCAACGAATTACAAGGGGAACACAAAAGGGGTTGTTTTATCCAGAGAATCAAAT |
| 7501 | AGTTTTCAATTTGAAGGATGGATACCAAGAATCCGTTTTACAAAAACTGAATTCTTAGAG |
| 7561 | GCTTATGGAGTTAAGCGGTATAAAACATCCAGAAATAAGTATGAGTTTAGTGGAAAAGAA |
| 7621 | GCTGAAACTGCTTTAGAAGCCTTATACCATTTAGGACATCAACCGTTTTTAATAGTGGCA |
| 7681 | ACTAGAACTCGATGGACTAATGGAACACAAATAGTAGACCGTTACCAAACTCTTTCTCCG |
| 7741 | ATCATTAGGATTTACGAAGGATGGGAAGGTTTAACTGACGAAGAAAATATAGATATAGAC |
| 7801 | TTAACACCTTTTAATTCACCATCTACACGGAAACATAAAGGGTTCGTTGTAGAGCCATGT |
| 7861 | CCTATCTTGGTAGATCAAATAGAATCCTACTTTGTAATCAAGCCTGCAAATGTATACCAA |
| 7921 | GAAATAAAAATGCGCTTCCCAAATGCATCAAAGTATGCTTACACATTTATCGACTGGGTG |
| 7981 | ATTACAGCAGCTGCGAAAAAGAGACGAAAATTAACTAAGGATAATTCTTGGCCAGAAAAC |
| 8041 | TTGTTCTTAAACGTTAACGTTAAAAGTCTTGCATATATTTTAAGGATGAATCGGTACATT |
| 8101 | TGTACAAGGAACTGGAAAAAAATCGAGTTAGCTATCGATAAATGTATAGAAATCGCCATT |
| 8161 | CAGCTTGGTTGGTTATCTAGAAGAAAACGCATTGAATTTCTGGATTCTTCTAAACTCTCT |
| 8221 | AAAAAAGAAATTCTATATCTAAATAAAGAGCGTTTTGAAGAAATAACTAAGAAATCTAAA |
| 8281 | GAACAAATGGAACAATTAGAACAAGAATCTATTAATTAATAGCAAACTTGAAACTAAAAA |
| 8341 | CCTAATTTATTTAAAGCTCAAAATAAAAAAGAGTTTTAAAATGGGAAATTCTGGTTTTTA |
| 8401 | TTTGTATAACACTCAAAACTGCGTCTTTGCTGATAATATCAAAGTTGGGCAAATGACAGA |
| 8461 | GCCGCTCAAGGACCAGCAAATAATCCTTGGGACAACATCAACACCTGTCGCAGCCAAAAT |
| 8521 | GACAGCTTCTGATGGAATATCTTTAACAGTCTCCAATAATCCATCAACCAATGCTTCTAT |
| 8581 | TACAATTGGTTTGGATGCGGAAAAAGCTTACCAGCTTATTCTAGAAAAGTTGGGAGATCA |
| 8641 | AATTCTTGGTGGAATTGCTGATACTATTGTTGATAGTACAGTCCAAGATATTTTAGACAA |
| 8701 | AATCACAACAGACCCTTCTCTAGGTTTGTTGAAAGCTTTTAACAACTTTCCAATCACTAA |
| 8761 | TAAAATTCAATGCAACGGGTTATTCACTCCCAGGAACATTGAAACTTTATTAGGAGGAAC |
| 8821 | TGAAATAGGAAAATTCACAGTCACACCCAAAAGCTCTGGGAGCATGTTCTTAGTCTCAGC |
| 8881 | AGATATTATTGCATCAAGAATGGAAGGCGGCGTTGTTCTAGCTTTGGTACGAGAAGGTGA |
| 8941 | TTCTAAGCCCTACGCGATTAGTTATGGATACTCATCAGGCGTTCCTAATTTATGTAGTCT |
| 9001 | AAGAACCAGAATTATTAATACAGGATTGACTCCGACAACGTATTCATTACGTGTAGGCGG |
| 9061 | TTTAGAAAGCGGTGTGGTATGGGTTAATGCCCTTTCTAATGGCAATGATATTTTAGGAAT |
| 9121 | AACAAATACTTCTAATGTATCTTTTTTGGAGGTAATACCTCAAACAAACGCTTAAACAAT |
| 9181 | TTTTATTGGATTTTTCTTATAGGTTTTATATTTAGAGAAAAAAGTTCGAATTACGGGGTT |
| 9241 | TGTTATGCAAAATAAAAGCAAAGTGAGGGACGATTTTATTAAAATTGTTAAAGATGTGAA |
| 9301 | AAAAGATTTCCCCGAATTAGACCTAAAAATACGAGTAAACAAGGAAAAAGTAACTTTCTT |
| 9361 | AAATTCTCCCTTAGAACTCTACCATAAAAGTGTCTCACTAATTCTAGGACTGCTTCAACA |
| 9421 | AATAGAAAACTCTTTAGGATTATTCCCAGACTCTCCTGTTCTTGAAAAATTAGAGGATAA |
| 9481 | CAGTTTAAAGCTAAAAAAGGCTTTGATTATGCTTATCTTGTCTAGAAAAGACATGTTTTC |
| 9541 | CAAGGCTGAATAGATAACTTACTCTAACGTTGGAGTTGATTTGCACACCTTAGTTTTTTG |
| 9601 | CTCTTTTAAGGGAGGAACTGGAAAAACAACACTTTCTCTAAACGTGGGATGCAACTTGGC |
| 9661 | CCAATTTTTAGGGAAAAAAGTGTTACTTGCTGACCTAGACCCGCAATCCAATTTATCTTC |
| 9721 | TGGATTGGGGGCTAGTGTCAGAAGTAACCAAAAAGGCTTACACGACATAGTATACACATC |
| 9781 | AAACGATTTAAAATCAATCATTTGCGAAACAAAAAAAGATAGTGTGGACCTAATTCCTGC |
| 9841 | ATCATTTTTATCCGAACAGTTTAGAGAATTGGATATTCATAGAGGACCTAGTAACAACTT |
| 9901 | AAAGTTATTTCTGAATGAGTACTGCGCTCCTTTTTATGACATCTGCATAATAGACACTCC |
| 9961 | ACCTAGCCTAGGAGGGTTAACGAAAGAAGCTTTTGTTGCAGGAGACAAATTAATTGCTTG |
| 10021 | TTTAACTCCAGAACCTTTTTCTATTCTAGGGTTACAAAAGATACGTGAATTCTTAAGTTC |
| 10081 | GGTCGGAAAACCTGAAGAAGAACACATTCTTGGAATAGCTTTGTCTTTTTGGGATGATCG |
| 10141 | TAACTCGACTAACCAAATGTATATAGACATTATCGAGTCTATTTACAAAAACAAGCTTTT |
| 10201 | TTCAACAAAAATTCGTCGAGATATTTCTCTCAGCCGTTCTCTTCTTAAAGAAGATTCTGT |
| 10261 | AGCTAATGTCTATCCAAATTCTAGGGCCGCAGAAGATATTCTGAAGTTAACGCATGAAAT |
| 10321 | AGCAAATATTTTGCATATCGAATATGAACGAGATTACTCTCAGAGGACAACGTGAACAAA |
| 10381 | CTAAAAAAAGAAGCGAATGTCTTTTTTAAAAAAAATCAAACTGCCGCTTCTTTAGATTTT |
| 10441 | AAGAAGACGCTTCCTTCCATTGAACTATTCTCAGCAACTTTGAATTCTGAGGAAAGTCAG |
| 10501 | AGTTTGGATCAATTATTTTTATCAGAGTCCCAAAACTATTCGGATGAAGAATTTTATCAA |
| 10561 | GAAGACATCCTAGCGGTAAAACTGCTTACTGGTCAGATAAAATCCATACAGAAGCAACAC |
| 10621 | GTACTTCTTTTAGGAGAAAAAATCTATAATGCTAGAAAAATCCTGAGTAAGGATCACTTC |
| 10681 | TCCTCAACAACTTTTTCATCTTGGATAGAGTTAGTTTTTAGAACTAAGTCTTCTGCTTAC |
| 10741 | AATGCTCTTGCATATTACGAGCTTTTTATAAACCTCCCCAACCAAACTCTACAAAAAGAG |
| 10801 | TTTCAATCGATCCCCTATAAATCCGCATATATTTTGGCCGCTAGAAAAGGCGATTTAAAA |
| 10861 | ACCAAGGTCGATGTGATAGGGAAAGTATGTGGAATGTCGAACTCATCGGCGATAAGGGTG |
| 10921 | TTGGATCAATTTCTTCCTTCATCTAGAAACAAAGACGTTAGAGAAACGATAGATAAGTCT |
| 10981 | GATTCAGAGAAGAATCGCCAATTATCTGATTTCTTAATAGAGATACTTCGCATCATGTGT |
| 11041 | TCCGGAGTcTCTTTGcCCTCCTATAACGAAAATCTTCTACAACAGCTTTTTGAACTTTTT |
| 11101 | AAGCAAAAGAGCTGATCCTCCGTCAGCTCATATATATATCTATTATATATATATATTTAG |
| 11161 | GGATTTGATTTTACGAGAGAGATTTGCAACTCTTGGTGGTAGACTTTGCAACTCTTGGTG |
| 11221 | GTAGACTTTGCAACTCTTGGTGGTAGACTTTGCAACTCTTGGTGGTAGACTTGGTCATAA |
| 11281 | TGGACTTTTGTTGAAAAATTTCTTAAAATCTTAGAGCTCCGATTTTGAATAGCTTTGGTT |
| 11341 | AAGAAAATGGGCTCGATGGCTTTCCATAAAAGTAGGTTGTTCTTAACTTTTGGGGACGCG |
| 11401 | TCGGAAATTTGGTTATCTACTTTATCTCATCTAACTAGAAAAAATTATGCGTCTGGGATT |
| 11461 | AACTTTCTTGTTTCTTTAGAGATTCTGGATTTATCGGAAACCTTGATAAAGGCTATTTCT |
| 11521 | CTTGACCACAGCGAATCTTTGTTTAAAATCAAGTCTCTAGATGTTTTTAATGGAAAAGTC |
| 11581 | GTTTCAGAGGCCTCTAAACAGGCTAGAGCGGCATGCTACATATCTTTCACAAAGTTTTTG |
| 11641 | TATAGATTGACCAAGGGATATATTAAACCCGCTATTCCATTGAAAGATTTTGGAAACACT |
| 11701 | ACATTTTTTAAAATCCGAGACAAAATCAAAACAGAATCGATTTCTAAGCAGGAATGGACA |
| 11761 | GTTTTTTTTGAAGCGCTCCGGATAGTGAATTATAGAGACTATTTAATCGGTAAATTGATT |
| 11821 | GTACAAGGGATCCGTAAGTTAGACGAAATTTTGTCTTTGCGCACAGACGATCTATTTTTT |
| 11881 | GCATCCAATCAGATTTCCTTTCGCATTAAAAAAAGACAGAATAAAGAAACCAAAATTCTA |
| 11941 | ATCACATTTCCTATCAGCTTAATGGAGGAGTTGCAAAAATACACTTGTGGGAGAAATGGG |
| 12001 | AGAGTATTTGTTTCTAAAATAGGGATTCCTGTAACAACAAGTCAGGTTGCGCATAATTTT |
| 12061 | AGGCTTGCAGAGTTCTATAGTGCTATGAAAAAAAAATTACTCCTAGAGTACTTCGTGCAA |
| 12121 | GCGCTTTGATTCATTTAAAGCAAATAGGATTAAAAGATGAGGAAATCATGCGTATTTCCT |
| 12181 | GTCTTTCATCGAGACAAAGTGTGTGTTCTTATTGTTCTGGGTGTCGACATTCTTGAACGG |
| 12241 | TGGAGACGGTTTCTTATAA TGACACCGACTT |

### B. Vectors

pTRL2-grgA-67m, which carried a grgA allele with resistance to intron insertion between nucleotides 67 and 68, was constructed by assembling 3 DNA fragments using the NEBuilder HiFi DNA assembly kit (New England Biolabs). All 3 fragments were amplified from pTRL2-His-GrgA using Q5 DNA polymerase (New England Biolabs™). Fragment 1 was generated using primers pgp3-pgp4-F and His-RBS-R (Table 1, above). Fragment 2 was generated using primers RBS-His-F and GrgA-67-R (Table 1, above). Fragment 3 was generated using primers GrgA-67-F and pgp4-pgp3-R (Table 1, above).

pDFTT3(aadA), a Targetron vector for disrupting chlamydial genes using group II intron mutagenesis, was obtained from Dr. Derek Fisher (Southern Illinois University, IL). To construct pDFTT3(aadA)-GrgA-67, designed for disrupting the open reading frame of grgA, two PCR fragments were first generated using pDFTT3(aadA) as the template. Fragment 1 was obtained using primers GrgA67_IBS1/2 and the University primers (Table 1, above), while fragment 2 was obtained using primers GrgA67_EBS2 and GrgA67_EBS1/delta (Table 1, above). The two fragments were combined and subject to PCR extension. The resulting full-length intron-targeting fragment was digested with HindIII and BsrGI and subjected to ligation with HindIII- and BsrGI-digested pDFTT3(aadA). The ligation product was transformed into E. coli DH5α, which was plated onto LB agar plates containing 500 μg/ml spectinomycin and 25 μg chloramphenicol. Authenticity of the insert in pDFTT3(aadA)-grgA-67m was confirmed using Sanger sequencing.

### C. Host Cells and Culture Conditions

Mouse fibroblast L929 cells were used as the host cells for C. trachomatis transformation and preparation of EBs. Unless indicated otherwise, human vaginal carcinoma HeLa cells were used for experiments determining the effects of GrgA depletion on chlamydial growth and development. Both L929 and HeLa cell lines were maintained as monolayer cultures using Dulbecco's modified Eagle's medium (DMEM) (Sigma Millipore™) containing 5% and 10% fetal bovine serum (vol/vol), respectively. Gentamicin (final concentration: 20 μg/mL) was used for maintenance of uninfected cells and was replaced with penicillin (10 units/mL) and/or spectinomycin (500 μg/mL) as detailed below. Incubators at 37° C., 5% CO₂ were used for culturing uninfected and infected cells.

### D. Chlamydiae

Wildtype C. trachomatis L2 434/BU (L2) was purchased from ATCC. This strain was chosen because 1) it is the best-studied model organism, 2) its genome is nearly identical to those of serovars with tropism for genital epithelial cells, 3) it is easy to grow in cell culture, and 4) nearly all genetics tools have been developed using this organism. Chlamydial strains also contemplated for use in the invention include any Chlamydia or Chlamydophila species.

L2/cg-peig was derived by transforming L2 EBs with pTRL2-grgA-67m using calcium phosphate as previously described in the art. The transformation was inoculated onto L929 monolayer cells and selected with penicillin. L2/cgad-peig was derived by transforming L2/cg-peig with pDFTT3(aadA)-grgA-67m in the same manner. ATC was added to the cultures immediately after transformation to induce the expression of GrgA from pDFTT3(aadA)-grgA-67m. Twelve hours later, spectinomycin D (final concentration: 500 μg/ml) was added to the culture medium to initiate selection. L2/cgad-peig EBs were amplified using L929 cells and purified with ultracentrifugation through MD76 density gradients. Purified EBs were resuspended in sucrose-phosphate-glutamate (SPG) buffer; small aliquots were made and stored at −80° C. Unless indicated otherwise, cycloheximide was added to all chlamydial cultures (final cycloheximide concentration in media: 1 nM) to optimize chlamydia growth.

### E. Immunofluorescence Staining

Near-confluent HeLa monolayers grown on 6-well plates were inoculated with L2/cgad-peig at MOI of 0.3 inclusion-forming units. Following a 20-minute centrifugation at 900 g, cells were cultured at 37° C. in media containing either 0 or 1 nM ATC for 30 hours. The infected cells were then fixed with cold methanol, blocked with 10% fetal bovine serum prepared in phosphate-buffered saline (PBS), and stained successively with the monoclonal L21-5 anti-major outer membrane protein antibody and an FITC-conjugated rabbit anti-mouse antibody. Immunostained cells finally were counter-stained with 0.01% Evan blue (in PBS) before imaging under an Olympus™ IX51 fluorescence microscope. Red and green fluorescence images were acquired on an Olympus™ IX51 fluorescence microscope using a constant exposure time for each channel. Image overlay was performed using the PictureFrame™ software. The Java-based ImageJ™ software was then used to process the image.

### F. IFU Assays

L2/cgad-peig EB stock or frozen harvests of L2/cgad-peig cultured with or without ATC were thawed, 1-to-10 serially diluted, and inoculated onto L929 monolayers in medium containing 1 nM ATC and 1 μg/mL cycloheximide on a 96-w plates. Following 20 minutes of centrifugation at 900 g, cells were cultured at 37° C. for 30 hours. Cell fixation and antibody reactions were performed as described above. Immunostained inclusions were counted under the fluorescence microscope without Evan blue counter staining.

### G. Diagnostic PCR and DNA Sequencing

For confirming and sequencing grgA alleles in the chromosome and plasmid, total RNA was extracted from about 1000 infected cells using the Quick-gDNA MiniPrep™ kit (Sigma Millipore™) following manufacturer's instructions. The resulting DNA was used for PCR amplification using the Taq DNA polymerase. DNA fragments resolved with electrophoresis using a 1.2% Agarose gel were exercised and purified using the Gel Extraction kit (Qiagen™) and subject to Sanger sequencing service provided by the Psomagen™ Service Center (New York).

### H. Quantitation of Genome Copy Numbers

To quantify genome copy numbers in cultures, infected cells on 6-, 12-, or 24-well plates were detached from the plastic using Cell Lifters (Corning™). Cells and media were collected into Eppendorf™ tubes and centrifuged at 20,000 g at 4° C. The supernatant was carefully aspirated. One hundred microliters of alkaline lysis buffer (100 mM NaOH and 0.2 mM EDTA) was added into each tube to dissolve the cell pellets. Tubes were heated at 95° C. for 15 minutes and then placed onto ice. Four hundred microliters of 50 mM Tri-HCl (7.0) was added into each tube. The neutralized extracts are used for qPCR analysis directly (1 μL/reaction) or after dilution with H₂O.

### I. Western Blotting

Detection of MOMP and GrgA was performed as described in the art. L929 cells grown on 6-well plates were infected with L2/cgad-peig and cultured with medium containing 1 nM ATC. At 15 hours, 15 hours 20 minutes, and 15 hours 40 minutes postinoculation, cells in selected wells were switched to ATC-free medium after 3 washes. At 16 hours post infection (hpi), cells in each well were harvested in 200 μL of 1×SDS-PAGE sample buffer, heated at 95° C. for 5 minutes, and sonicated for 1 minute (5 seconds on/5 seconds off) at 35% amplitude. Proteins were resolved in 10% SDS-PAGE gels and thereafter transferred onto PVDF membranes. The membrane was probed with the monoclonal mouse anti-MOMP MC22 antibody, stripped and reprobed with a polyclonal mouse anti-GrgA antibody.

### J. Transmission Electron Microscopy

To visualize intracellular chlamydiae up to 36 hpi, L929 cell monolayers grown on 6-well plates were infected as described above and cultured with medium supplemented with or without 1 nM ATC. For cultures up to 36 hours, cells were removed from the plastic surface using trypsin, collected in PBS containing 10% fetal bovine serum, and centrifuged for 10 minutes at 500 g. Pelleted cells were resuspended in EM fixation buffer (2.5% glutaraldehyde, 4% paraformaldehyde, 0.1 M cacodylate buffer) at RT, allowed to incubate for 2 hours, and stored at 4° C. overnight. To visualize intracellular chlamydiae at 45 and 60 hpi, the above procedures resulted in lysis of infected cells and inclusions. To overcome this problem, cells grown on glass coverslips were infected with and fixed without trypsinization. To prepare samples for imaging, cells were first rinsed in 0.1 M cacodylate buffer, dehydrated in a graded series of ethanol, and then embedded in Eponate 812 resin at 68° C. overnight. Ninety nanometer thin sections were cut on a Leica™ UC6 microtome and picked up on a copper grid. Grids were stained with Uranyl acetate followed by Lead Citrate. TIFF images were acquired on a Philips™ CM12 electron microscope at 80 kV using an AMT XR111 digital camera. RB diameters were measured using ImageJ™ software.

### Example 2: MOMP Immunostaining, qPCR Analysis, and Detection of Progeny EBs

MOMP immunostaining was performed as described in Example 1. The results showed significantly smaller inclusions in ATC-free cultures, compared with 1.0 nM ATC cultures, at 34 hpi.

### Example 3: Dependence on Plasmid-Expressed (DOPE) Gene Technology

The DOPE technology was developed to investigate the biological functions and underlying mechanisms of genes essential for chlamydial growth and/or development.

1. DOPE for Targetron (group II intron) disruption

1.1. Construct an inducible GrgA expression vector (GrgA DOPE plasmid)

1.1a. Use the plasmid from the Chlamydia spp. that DOPE is intended for.

1.1b. Synonymously mutate the intron target sites so that the plasmid-carried essential gene is resistant to intron-targeting vector. This step increases the efficiency of selecting chlamydiae with the essential gene disrupted by the intron in the chromosome but may not be essential. Group II intron target sites can be identified using the Targetron algorithm.

1.1c. Use an appropriate inducible expression system. The inducible system could be controlled by anhydrotetracycline or one of derivatives, isopropyl β-d-1-thiogalactopyranoside (IPTG), theophylline, etc.

1.2. Transform chlamydia with the inducible expression vector

1.2a. Mix the GrgA expression vector with chlamydial EBs and calcium phosphate prepared in HEPES buffer.

1.2b. Inoculate the above mix onto host cells.

1.2c. Select for transformants with an appropriate antibiotic.

1.3. Construct Targetron mutagenesis vector

1.3a. Use a different antibiotic resistance gene from one used for the GrgA expression vector.

1.3b. Use the Targetron algorithm to facilitate the construction.

1.4. Transform DOPE plasmid-transformed Chlamydia with the GrgA-targeting Targetron vector

1.4a. Perform transformation and infection as steps 1.2a and 1.2b.

1.4b. Select for transformants using medium containing the antibiotic that selects for the insertion of group II intron and ATC to induce GrgA expression from the DOPE plasmid.

1.4c. Confirm disruption of the chromosomal grgA and the intactness of plasmid grgA using PCR analysis and DNA sequencing.

1.5. Determine GrgA dependence

1.5a. Prepare EBs from cultures containing ATC. Remove ATC from the stocks by washes with a buffer (e.g., sucrose-phosphate-glutamate solution).

1.5b. Inoculate cells with ATC-free EBs prepared in the preceding step.

1.5c. Culture infected cells in ATC-free medium and ATC-containing medium.

1.5d. Evaluate dependency through different means including but not limited to analysis of inclusion size, genome replication, and EB production.

2. DOPE for knocking out using homologous recombination

2.1. Construct an inducible GrgA expression vector (GrgA DOPE plasmid) Construct the DOPE plasmid as described under 1.1a and 1.1c. Disregard point 1.1b.

2.2. Transform chlamydia with the inducible GrgA expression vector Preform transformation and selection as described under 1.2.

2.3. Construct vector for homologous recombination

Construct a plasmid carrying the sequences flanking the coding regions of grgA. Replace the entirety or part of the open reading frame of grgA with an antibiotic selection marker, which must be different from the one in the DOPE plasmid.

2.4. Transform DOPE plasmid-transformed Chlamydia with the GrgA-targeting vector Perform transformation, selection, and confirmation of as described under 1.4. Replace the Targetron vector (step 1.3) made the one made in step 2.3.

2.5. Determine GrgA dependence

Prepare ATC-free EB stocks, infection, culture and characterization as described in steps under 1.5.

3. DOPE for knocking out using CRISPR

3.1. Construct an inducible GrgA expression vector (GrgA DOPE plasmid)

Construct the DOPE plasmid as described under 2.1.

3.2. Transform chlamydia with the inducible GrgA expression vector

Preform transformation and selection as described under 1.2.

3.3. Construct vector for homologous recombination

Construct a grgA-targeting CRISPR knock-out vector, which contains a CRISPR-associated (CAS) gene, grgA-targeting guide RNA, and antibiotic-resistant gene carrying sequencing flanking the CRISPR-target site. The antibiotic selection marker in the CRIPSR-targeting vector must be different from the one in the DOPE plasmid.

3.4. Transform DOPE plasmid-transformed Chlamydia with the GrgA-targeting vector Perform transformation, selection, and confirmation of as described under 1.4. Replace the Targetron vector (step 1.3) made the one made in step 3.3.

3.5. Determine GrgA dependence

Prepare ATC-free EB stocks, infection, culture and characterization as described in steps under 1.5.

In summary, the grgA gene was disrupted in the Chlamydia chromosome using a group II intron bearing aadA, which confers resistance to spectinomycin. The disruption was made possible only in Chlamydia transformed with a plasmid carrying grgA, in which the intron target site was synonymously mutated. Hence, this gene-targeting strategy is referred to as DOPE (dependence on plasmid-mediated expression). The ATC-inducible expression system was reengineered in the DOPE plasmid to drastically diminish the GrgA expression level, which allowed for effective complementation of chromosomal grgA disruption without excessive GrgA overexpression-mediated growth inhibition.

Targetron™, a group II intron-based insertional mutagenesis technology, has been used to successfully disrupt numerous chlamydial chromosomal genes. So, in an effort to knock out GrgA expression in Chlamydia and investigate its physiological actions, Targetron™ vectors containing spectinomycin-resistance gene-bearing group II introns specific for multiple grgA insertion sites. Despite several attempts, we failed to generate any grgA-null mutants. Spectinomycin-resistant chlamydiae were obtained from only two transformed cultures, yet diagnostic PCR analysis failed to demonstrate insertion of the group II intron into grgA indicating nonspecific targeting. Together with the failure to obtain grgA-null mutants, failure to establish stable grgA-null mutants using Targetron™ insertional mutagenesis suggested to us that grgA was essential for chlamydial growth and development. As a new and alternative approach to investigate the biological functions and underlying mechanisms of grgA and other genes essential for chlamydial growth and/or development, we developed the dependence on plasmid-mediated expression (DOPE) tool (see FIG. 3). Because essential genes are required for normal chlamydial growth or development, their disruption in wildtype chlamydiae will result in lethality (FIG. 3A and FIG. 3B). However, transformation of wildtype Chlamydia with a recombinant plasmid carrying the essential chromosomal gene downstream of an inducible promoter allows for the disruption of the chromosomal allele when the inducer is present in the culture medium (FIG. 3C). In the resulting essential gene-disrupted strain, withdrawal of the inducer causes depletion of the gene products generated from the recombinant plasmid and allow for functional and mechanistic analyses of the essential gene (FIG. 3D).

### Example 4: Mechanistic Interrogation of Chromosome-Encoded Essential Genes

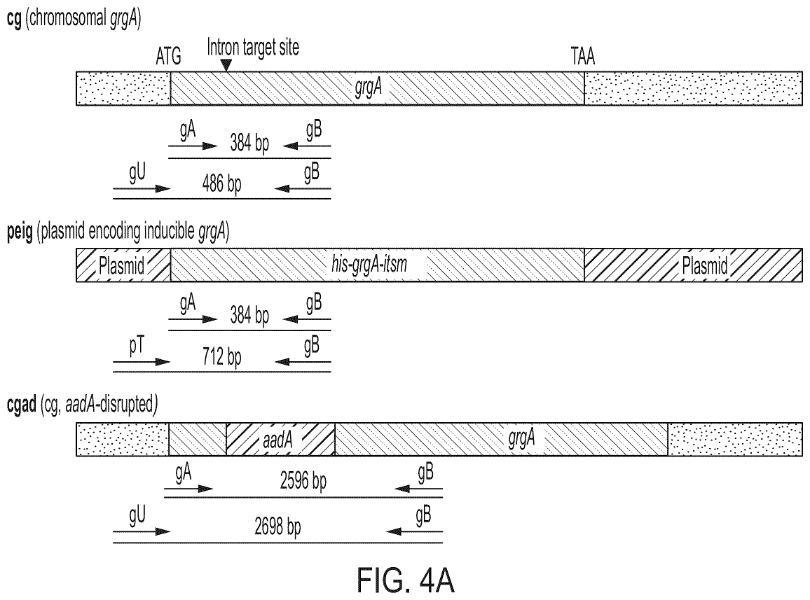

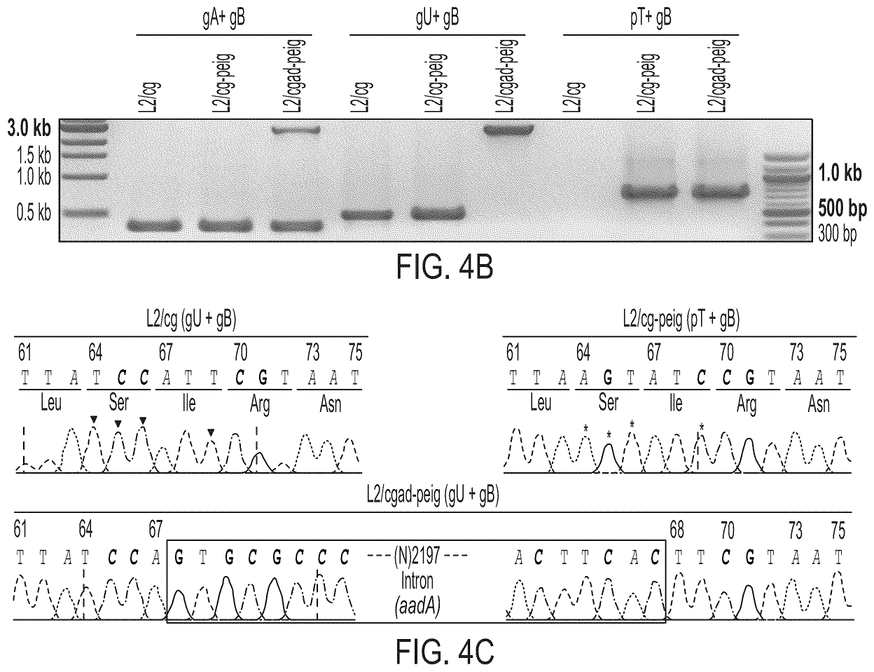

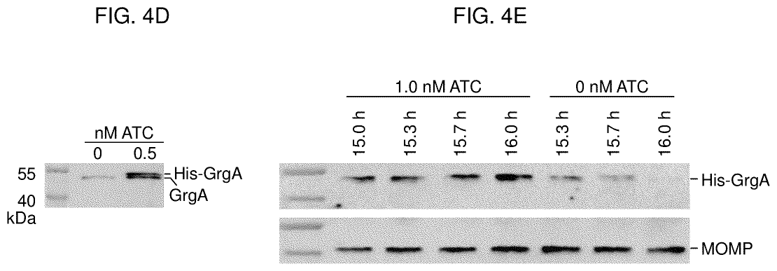

To apply DOPE to studying grgA, a plasmid, pTRL2-peig, which encodes an anhydrotetracycline (ATC)-inducible grgA allele (i.e., peig) was constructed. See the sequences provided above. Compared to the native chromosomal grgA allele that contains a group II intron-target site between nucleotides 67 and 68, the grgA allele in peig carried a His-tag sequence and four synonymous point mutations surrounding the group II intron-targeting site. See FIG. 4A. We transformed wildtype C. trachomatis with an intact chromosomal grgA (L2/cg) with peig to derive L2/cg-peig.

We next transformed L2/cg-peig with the aforementioned Targetron™ plasmid carrying an aadA-containing group II intron with the insertion site between nucleotides 67 and 68 in grgA. Since the Targeton™ target site in the peig allele has been mutated, the vector can only insert into the chromosomal grgA allele (FIG. 4A). Following transformation of L2/cg-peig with the Targetron™ plasmid, we supplemented the culture medium with ATC and spectinomycin to induce GrgA expression from peig while selecting for chromosomal mutants carrying an intron-disrupted grgA.

PCR analysis confirmed the chlamydial genotypes L2/cg, L2/cg-peig, as well as the plasmid-complemented, chromosomal grgA-disrupted L2/cgad-peig. See FIG. 4A and FIG. 4B. Tracings of Sanger sequences in FIG. 4C confirmed the nucleotide sequences surrounding the intron-target site in L2/cg and L2/cg-peig and the grgA-intron joint regions in L2/cgad-peig. As expected, western blotting detected ATC-independent chromosome-encoded GrgA expression and ATC-dependent plasmid-encoded His-GrgA expression in L2/cg-peig. See FIG. 4D. Significantly, western blotting also detected time-dependent GrgA loss in L2/cgad-peig upon ATC withdrawal, as shown in FIG. 4E. By 2 hours post-ATC withdrawal, His-GrgA became nearly undetectable. Taken together, the data presented in FIG. 4B through FIG. 4E demonstrate that wildtype chromosomal grgA was successfully disrupted in L2/cgad-peig, Expression, and thus function, of GrgA therefore depends on ATC-induced GrgA expression from the peig plasmid.

In FIG. 4, dark and grey lines signify chromosomal and plasmid sequences, respectively. Half arrows indicate transcription. EG: essential genes; iEG: inducible EG; chr: chromosome; NA: not applicable; g: growth; d: development. The figure shows confirmation of the disruption of the chromosome-encoded grgA by group II-intron and re-expression of GrgA from a transformed plasmid in the DOPE system. FIG. 4A presents schematic drawings of grgA alleles, locations of intron-target site, diagnostic primers, and size of PCR products obtained with different sets of primers. Abbreviations: itsm, intron target site mutated; Chr, chromosome. FIG. 4B provides a gel image of PCR products amplified with DNA of wildtype C. trachomatis L2 (L2/cg), L2/cg transformed with the his-grgA-itsm expression plasmid (L2/cg-peig), and L2 with aadA-disrupted chromosomal grgA complemented with peig (L2/cgad-peig) using the primer sets shown in FIG. 4A. FIG. 4C presents Sanger sequencing tracings of PCR products showing the intron-target site in L2/cg, mutations surrounding this site conferring resistance to intron targeting in peig, and grgA-intron joint regions in the chromosome of L2/cg-peig. Wildtype bases and corresponding mutated bases are shown with arrowheads and asterisks, respectively. FIG. 4D shows western blotting detection only chromosome-encoded GrgA in L2/cg-peig cultured in ATC-free medium and both chromosome-encoded GrgA and plasmid-encoded His-GrgA in L2/cg-peig cultured in the ATC-containing medium for 14 hours. FIG. 4E shows western blotting showing time-dependent loss of His-GrgA in L2/cgad-peig upon ATC withdrawal. The membrane was first probed with an anti-major outer membrane protein, stripped, and then reprobed with an anti-GrgA antibody.

Compared to the native chromosomal grgA allele that contains a group II intron-target site between bases 67 and 68, the grgA allele in peig carried a His-tag-encoding sequence and four synonymous point mutations surrounding the group II intron-targeting site. See FIG. 4A. Wildtype C. trachomatis (i.e., L2/cg) was transformed with plasmid pTRL2-peig to derive L2/cg-peig. L2/cg-peig next was transformed with a suicidal plasmid, pTargetron-aadA-grgA67, carrying an aadA-containing group II intron targeting bases 67 and 68 of grgA (see FIG. 4A). Since the target site in peig has been mutated, the intron can insert only to the cg allele (see FIG. 4A).

Following the transformation of L2/cg-peig with the intron plasmid, the culture medium was supplemented with 1 nM ATC and 500 μg/mL spectinomycin to induce GrgA expression from peig and to select for chlamydiae with intron(aadA)-disrupted grgA, respectively. PCR analysis (see FIG. 4B) confirmed the genotype L2/cg, L2/cg-peig, as well as the plasmid-complemented, chromosomal grgA-disrupted L2/cgad-peig (FIG. 4B) using the primers shown in FIG. 4A. The tracings of Sanger sequences in FIG. 4C confirm the nucleotide sequences surrounding the intron-target site in L2/cg, L2/cg-peig, and the grgA-intron joint regions in L2/cgad-peig. As expected, western blotting detected ATC-independent chromosome-encoded GrgA expression and ATC-dependent plasmid-encoded His-GrgA expression in L2/cg-peig. See FIG. 4D. Significantly, western blotting also detected time-dependent GrgA loss in L2/cgad-peig upon ATC withdrawal. The membrane was first probed with an anti-major outer membrane protein (MOMP), stripped and then re-probed with an anti-GrgA antibody. See Example 1 for methods. By 2 hours after ATC withdrawal, His-GrgA became nearly undetectable. See FIG. 4E. Taken together, data presented in FIG. 4B through FIG. 4E demonstrate that grgA was successfully disrupted in the chromosome of L2/cgad-peig, in which expression and thus the function of GrgA depends on the presence of ATC in the culture medium.

### Example 5: tetR Mutations Enable GrgA Expression and EBs to Escape in the Absence of ATC

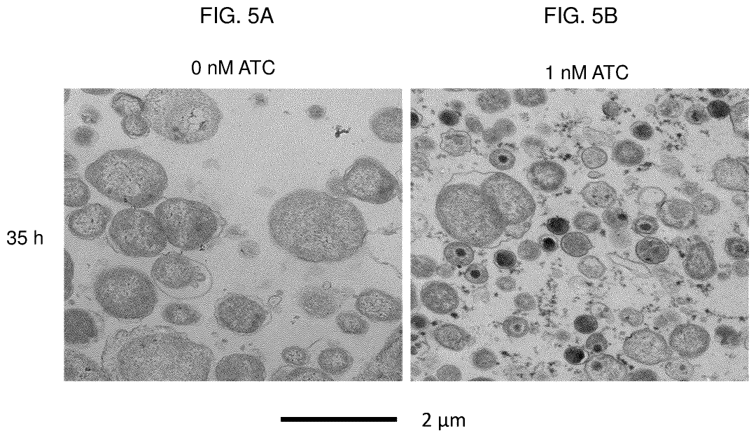

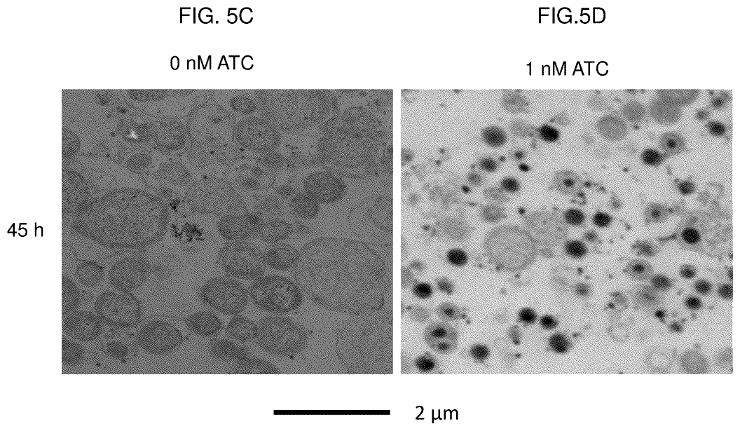

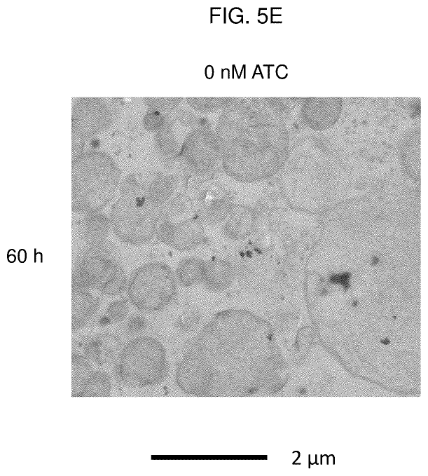

Consistent with the EB quantification assays, ultra-thin section transmission electron microscopy readily detected EBs at 36 hours in the ATC-containing cultures and were predominant at 45 hours. See FIG. 5. In contrast, EBs were rarely found in ATC-free cultures at 35, 45, or 60 hours. See FIG. 5. Taken together, these findings demonstrate that GrgA deficiency plays an important role in optimal RB growth and is crucial for the production of infectious EBs from RBs. The electron microscopy demonstrates lack of EBs in L2/cgad-peig cultured in ATC-free medium at 35, 45, and 60 hours. One nanomolar ATC cultures at 60 hours were not processed for EM because most inclusions already had burst by that point. Note that EBs are approximately 400 nm diameter cellular forms with high electron density found in ATC-containing cultures. The small irregularly-shaped dark particles in both ATC-containing and ATC-free cultures are glycogen particles.

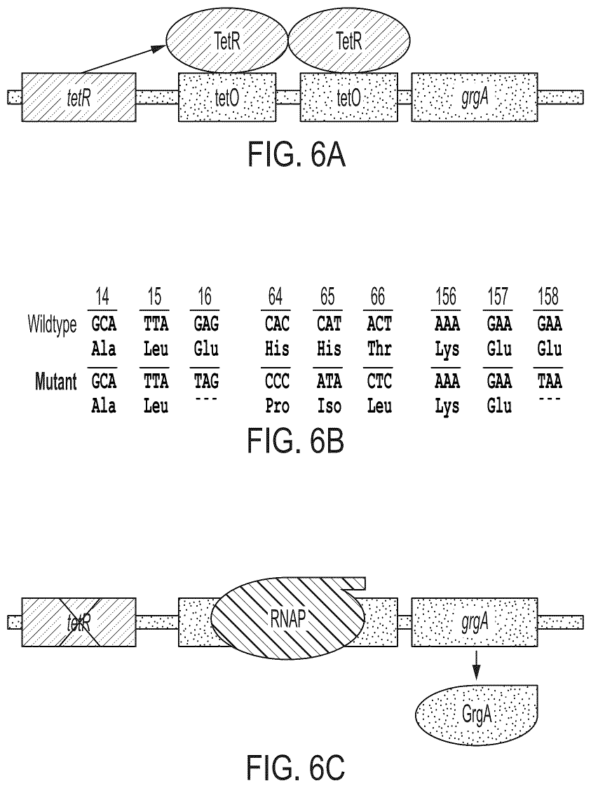

### Example 6: EB Escape in ATC-Free Cultures of L2/cgad-peig

Whereas GrgA-null chlamydiae display a severe deficiency in the formation of EBs, we were able to detect an extremely low trace background of EBs in chlamydial cultures lacking ATC. Since mutations in the tetR gene and/or mutations in tetO (TetR operator) might disable the ability of TetR to repress grgA in L2/cgad-peig and consequently allow EBs to form in the absence of ATC, we recovered plasmids from EBs formed in ATC-free cultures and expanded them in E. coli. Notably, DNA sequencing showed that a single nucleotide polymorphism (SNP) in the tetR gene had occurred in all 10 plasmids. Three distinct SNPs were detected; their locations and effects on the 208-aa TetR protein are shown in FIG. 6. Two of the SNPs cause premature termination at codons 16 or 158, while the third causes a frame shift at codon 64. These findings indicate that lack of full-length TetR, or synthesis of a functionally defective TetR, results in the leaky expression of wildtype GrgA in the absence of ATC.

For FIG. 6A, in L2/cgad-peig, the plasmid allele of grgA is repressed in the presence of ATC. For FIG. 6B, mutations identified in tetR in the plasmids isolated from L2/cg-peig EBs formed in the absence of ATC lead to premature translation termination or frameshift. Codon positions are indicated. Wild type DNA and amino acids are shown in black and mutated or frame-shifted nucleotides as well as consequent translational effects in red. For FIG. 6C, the mutations in FIG. 6B causes a loss in grgA repression, which enables peig to express functional TetR and consequent EB formation.

The data presented here leads to the conclusion that GrgA is required for EB formation.

### REFERENCES

All references listed below and throughout the specification are hereby incorporated by reference in their entirety.
1. Abdelrahman, Y. M. & Belland, R. J. The chlamydial developmental cycle. FEMS Microbiol Rev 29, 949-959, doi:S0168-6445(05)00023-9 [pii]10.1016/j.femsre.2005.03.002 (2005).
2. Abraham, S., Juel, H. B., Bang, P., Cheeseman, H. M., Dohn, R. B., et al. Safety and immunogenicity of the chlamydia vaccine candidate CTH522 adjuvanted with CAFO1 liposomes or aluminum hydroxide: a first-in-human randomized, double-blind, placebo-controlled, phase 1 trial. The Lancet 19(10):P1091-1100 (2019).
3. Bao, X., Nickels, B. E. & Fan, H. Chlamydia trachomatis protein GrgA activates transcription by contacting the nonconserved region of σ⁶⁶. Proc Natl Acad Sci USA 109, 16870-16875, doi:10.1073/pnas.1207300109 (2012).
4. Bao, X., Gylfe, A., Sturdevant, Z. G., Xu, S., Caldwell, H. D., Elofsson, M., and Fan, H. Benzylidine acylhydrazides inhibit Chlamydial growth in a type III secretion- and iron chelation-independent manner. J. Bacteriol. 196(16):2989 (2014).
5. Belland, R. J. et al. Genomic transcriptional profiling of the developmental cycle of Chlamydia trachomatis. Proc Natl Acad Sci USA 100, 8478-8483, doi:10.1073/pnas.1331135100 (2003).
6. Caldwell, H. D., Kromhout, J. & Schachter, J. Purification and partial characterization of the major outer membrane protein of Chlamydia trachomatis. Infect Immun 31, 1161-1176 (1981).
7. Cosse, M. M. et al. The loss of expression of a single type 3 effector (CT622) strongly reduces Chlamydia trachomatis infectivity and growth. Front Cell Infect Microbiol 8, 145, doi:10.3389/fcimb.2018.00145 (2018).
8. Darst, S. A. Bacterial RNA polymerase. Current opinion in structural biology 11, 155-162 (2001).
9. Desai, M., Wurihan, W., Di, R., Fondell, J. D., Nickels, B. E., Bao, X. Role for GrgA in regulation of σ28-dependent transcription in the obligate intracellular bacterial pathogen Chlamydia trachomatis. J. Bacteriol. 200(20):15(2018).
10. Feklistov, A., Sharon, B. D., Darst, S. A. & Gross, C. A. Bacterial sigma factors: a historical, structural, and genomic perspective. Annu Rev Microbiol 68, 357-376, doi:10.1146/annurev-micro-092412-155737 (2014).
11. Garrood, W. T. et al. Analysis of off-target effects in CRISPR-based gene drives in the human malaria mosquito. Proc Natl Acad Sci USA 118, doi:10.1073/pnas.2004838117 (2021).
12. Hanson, B. R. & Tan, M. Transcriptional regulation of the Chlamydia heat shock stress response in an intracellular infection. Mol Microbiol 97, 1158-1167, doi:10.1111/mmi.13093 (2015).
13. Hiu Yin Yu, H. & Tan, M. Sigma28 RNA polymerase regulates hctB, a late developmental gene in Chlamydia. Mol Microbiol 50, 577-584 (2003).
14. Hybiske, K. & Stephens, R. S. Mechanisms of Chlamydia trachomatis entry into nonphagocytic cells. Infect Immun 75, 3925-3934, doi:10.1128/iai.00106-07 (2007).
15. Hybiske, K. & Stephens, R. S. Mechanisms of host cell exit by the intracellular bacterium Chlamydia. Proc Natl Acad Sci USA 104, 11430-11435, doi:10.1073/pnas.0703218104 (2007).
16. Johnson, C. M. & Fisher, D. J. Site-specific, insertional inactivation of incA in Chlamydia trachomatis using a group II intron. PLoS One 8, e83989, doi:10.1371/journal.pone.0083989 (2013).
17. Key, C. E. & Fisher, D. J. Use of group II Intron technology for targeted mutagenesis in Chlamydia trachomatis. Methods Mol Biol 1498, 163-177, doi:10.1007/978-1-4939-6472-7_11 (2017).
18. Lowden, N. M., Yeruva, L., Johnson, C. M., Bowlin, A. K. & Fisher, D. J. Use of aminoglycoside 3′ adenyltransferase as a selection marker for Chlamydia trachomatis intron-mutagenesis and in vivo intron stability. BMC Research Notes 8, 1-10, doi:10.1186/s13104-015-1542-9 (2015).
19. Lund, P. A. Microbial molecular chaperones. Adv Microb Physiol 44, 93-140, doi:10.1016/s0065-2911(01)44012-4 (2001).
20. Mueller, K. E., Wolf, K. & Fields, K. A. Gene deletion by fluorescence-reported allelic exchange mutagenesis in Chlamydia trachomatis. mBio 7, doi:10.1128/mBio.01817-15 (2016).
21. Nguyen, B. D. & Valdivia, R. H. Virulence determinants in the obligate intracellular pathogen Chlamydia trachomatis revealed by forward genetic approaches. Proc Natl Acad Sci USA 109, 1263-1268, doi:10.1073/pnas.1117884109 (2012).
22. Nobles, C. L. iGUIDE Method for CRISPR Off-Target Detection. Methods Mol Biol 2189, 71-80, doi:10.1007/978-1-0716-0822-7_6 (2021).
23. Ouellette, S. P. Feasibility of a conditional knockout system for Chlamydia based on CRISPR interference. Frontiers in Cellular and Infection Microbiology 8, 59, doi:10.3389/fcimb.2018.00059 (2018).
24. Lee, J. K. et al. Replication-dependent size reduction precedes differentiation in Chlamydia trachomatis. Nature communications 9, 45, doi:10.1038/s41467-017-02432-0 (2018).
25. Mooney, R. A., Darst, S. A. & Landick, R. Sigma and RNA polymerase: an on-again, off-again relationship? Mol Cell 20, 335-345, doi:10.1016/j.molcel.2005.10.015 (2005).
26. Nicholson, T. L., Olinger, L., Chong, K., Schoolnik, G. & Stephens, R. S. Global stage-specific gene regulation during the developmental cycle of Chlamydia trachomatis. J Bacteriol 185, 3179-3189, doi:10.1128/JB.185.10.3179-3189.2003 (2003).
27. Pal, S., Cruz-Fisher, M. I., Cheng, C., Camichael, J. R., Tifrea, D. F., Tatarenkova, O., and de la Maza, L. M. Vaccination with the recombinant major outer membrane protein elicits long-term protection in mice against vaginal shedding and infertility following a Chlamydia muridarum genital challenge. npj Vaccines 020-00239-7 (2020).
28. Read, T. D. et al. Genome sequences of Chlamydia trachomatis MoPn and Chlamydia pneumoniae AR39. Nucl. Acids. Res. 28, 1397-1406 (2000).
29. Rosario, C. J., Hanson, B. R. & Tan, M. The transcriptional repressor EUO regulates both subsets of Chlamydia late genes. Mol Microbiol 94, 888-897, doi:10.1111/mmi.12804 (2014).
30. Shen, L. et al. Selective promoter recognition by chlamydial sigma28 holoenzyme. J Bacteriol 188, 7364-7377, doi:10.1128/JB.01014-06 (2006).
31. Soules, K. R., LaBrie, S. D., May, B. H. & Hefty, P. S. Sigma 54-regulated transcription is associated with membrane reorganization and type III secretion effectors during conversion to infectious forms of Chlamydia trachomatis. mBio 11, doi:10.1128/mBio.01725-20 (2020).
32. Sixt, B. S. et al. The Chlamydia trachomatis Inclusion Membrane Protein CpoS Counteracts STING-Mediated Cellular Surveillance and Suicide Programs. Cell Host Microbe 21, 113-121, doi:doi.org/10.1016/j.chom.2016.12.002 (2017).
33. Skipp, P. J. et al. Quantitative Proteomics of the Infectious and Replicative Forms of Chlamydia trachomatis. PLoS One 11, e0149011, doi:10.1371/journal.pone.0149011 (2016).
34. Stephens, R. S. et al. Genome sequence of an obligate intracellular pathogen of humans: Chlamydia trachomatis. Science 282, 754-759 (1998).
35. Thomson, N. R. et al. Chlamydia trachomatis: Genome sequence analysis of lymphogranuloma venereum isolates. Genome Res. 18, 161-171, doi:10.1101/gr.7020108 (2008).
36. Thompson, C. C. et al. The Rsb phosphoregulatory network controls availability of the primary sigma factor in Chlamydia trachomatis and influences the kinetics of growth and development. PLoS Pathog 11, e1005125, doi:10.1371/journal.ppat.1005125 (2015).
37. Wang, Y. et al. Development of a transformation system for Chlamydia trachomatis: restoration of glycogen biosynthesis by acquisition of a plasmid shuttle vector. PLoS Pathog 7, e1002258, doi:10.1371/journal.ppat.1002258 (2011).
38. Weber, M. M. et al. A Functional Core of IncA Is Required for Chlamydia trachomatis Inclusion Fusion. J Bacteriol 198, 1347-1355, doi:10.1128/jb.00933-15 (2016).
39. Wickstrum, J., Sammons, L. R., Restivo, K. N. & Hefty, P. S. Conditional gene expression in Chlamydia trachomatis using the tet system. PLoS One 8, e76743, doi:10.1371/journal.pone.0076743 (2013).
40. Wood, N. A. et al. The ClpX and ClpP2 Orthologs of Chlamydia trachomatis Perform Discrete and Essential Functions in Organism Growth and Development. mBio 11, doi:10.1128/mBio.02016-20 (2020).
41. Wurihan, W., Huang, Y., Weber, A. M., Wu, X. & Fan, H. Nonspecific toxicities of Streptococcus pyogenes and Staphylococcus aureus dCas9 in Chlamydia trachomatis. Pathog Dis, ftaa005, doi:10.1093/femspd/ftaa005 (2020).
42. Wurihan, W. et al. GrgA overexpression inhibits Chlamydia trachomatis growth through sigma66- and sigma28-dependent mechanisms. Microb Pathog 156, 104917, doi:doi.org/10.1016/j.micpath.2021.104917 (2021).
43. Wurihan, W. et al. Identification of a GrgA-Euo-HrcA transcriptional regulatory network in Chlamydia. mSystems, e0073821, doi:10.1128/mSystems.00738-21 (2021).
44. Wurihan, W., Zou, Y., Weber, A. M., Weldon, K., Huang, Y., Gong, Z., Lou, Z., Sun, S., Zhu, C., Wu, X., Zhou, J., Wang, Y., Lai, Z., and Fan, H. GrgA controls Chlamydia trachomatis growth and development by 2 regulating expression of transcription factors Euo and HrcA. https://doi.org/10.1101/2020.07.08.184431 (2020).
45. Wurihan, W., Zou, Y., Weber, A. M., Weldon, K., Huang, Y., Bao, X., Wang, Y., Lai, Z., and Fan, H. Identification of a GrgA-Euo-HrcA transcriptional regulatory network in Chlamydia. mSystems, e00738-21, doi:10.1128/mSystems.00738-21 (2021).
46. Wurihan, W., Weber, A. M., Gong, Z., Lou, Z., Sun, S., Zhou, J., and Fan, H. GrgA overexpression inhibits Chlamydia trachomatis growth through sigma66- and sigma28-dependent mechanisms. Microbial Pathogenesis 156:104917 (2021).
47. Zhang, Y. X., Stewart, S., Joseph, T., Taylor, H. R. & Caldwell, H. D. Protective monoclonal antibodies recognize epitopes located on the major outer membrane protein of Chlamydia trachomatis. J Immunol 138, 575-581 (1987).
48. Zhang, Y. X., Stewart, S. J. & Caldwell, H. D. Protective monoclonal antibodies to Chlamydia trachomatis serovar- and serogroup-specific major outer membrane protein determinants. Infect Immun 57, 636-638 (1989).
49. Zhong, G., Fan, P., Ji, H., Dong, F. & Huang, Y. Identification of a chlamydial protease-like activity factor responsible for the degradation of host transcription factors. J Exp Med 193, 935-942 (2001).
50. Zhong, J., Karberg, M. & Lambowitz, A. M. Targeted and random bacterial gene disruption using a group II intron (targetron) vector containing a retrotransposition-activated selectable marker. Nucl Acids Res 31, doi:10.1093/nar/gkg248 (2003).
51. U.S. Pat. No. 10,258,282.
52. U.S. Pat. No. 10,925,954.
53. pubmed.ncbi.nlm.nih.gov/31416692.

1. A Chlamydia knock-out that does not express GrgA.

2. The Chlamydia knock-out of claim 1 wherein the Chlamydia is selected from the group consisting of C. trachomatis, C. pneumoniae, C. psittaci, C. muridarum, C. suis, Chlamydophila. abortus, Chlamydophila felis, Chlamydophila pecorum, C. ibidis, C. avium, C. gallinacea, and the like.

3. The Chlamydia knock-out of claim 2 wherein the Chlamydia is selected from the group consisting of C. trachomatis, C. pneumoniae, and C. psittaci.

4. A vaccine comprising the Chlamydia knock-out of claim 1 and a pharmaceutically acceptable carrier.

5. A method of stimulating an immune response to Chlamydia in a subject in need, comprising: administering the vaccine of claim 4 to the subject.

6. A method of producing neutralizing antibodies to Chlamydia in a subject in need, comprising: administering the vaccine of claim 4 to the subject.

7. A method of determining whether a bacterial gene in a bacterial cell is essential to its growth or development, comprising:
(a) determining the growth or development of the bacterial cell when the gene is intact and expressed;
(b) disrupting the gene and determining the growth or development of the bacterial cell when the gene is not functional;
(c) introducing into the bacterial cell with the bacterial gene disrupted, a plasmid that contains an inducible version of the bacterial gene and determining the growth or development of the bacterial cell when the bacterial gene is induced and when the bacterial gene is not induced.

8. The method of claim 7 wherein the bacterial cell is a chlamydia spp.
